(19) United States
(12) Patent Application Publication
(10) Pub. No.: US 2023/0279560 A1
(43) Pub. Date: Sep. 7, 2023

(54) CYCLIC COMPOUNDS AS AROMA CHEMICALS

(71) Applicant: BASF SE, Ludwigshafen am Rhein (DE)

(72) Inventors: Axel KIRSTE, Meckenheim (DE); Ralf PELZER, Lampertheim (DE); Nicola Christiane AUST, Ludwigshafen am Rhein (DE); Florian BIRK, Giessen (DE); Marco Alexander FRAATZ, Giessen (DE); Holger ZORN, Giessen (DE)

(21) Appl. No.: 18/017,680

(22) PCT Filed: Jul. 27, 2021
(86) PCT No.: PCT/EP2021/070983

§ 371 Date: Jan. 24, 2023

(30) Foreign Application Priority Data
Jul. 28, 2020 (EP) 20188087.9

(51) Int. Cl. C25B 3/07; C07C 43/188; C07C 43/162; C11B 9/00; C25B 3/29; C25B 3/23

(52) U.S. Cl. CPC C25B 3/07; C07C 43/188; C07C 43/162; C11B 9/0015; C25B 3/29; C25B 3/23; C07C 2601/16; C07C 2601/14; C07C 2601/08; C07C 2602/32

(57) ABSTRACT

The invention relates to the use of terpene C₁-C₈-alkyl ether compositions and novel C₁-C₈-alkyl ethers of mono- and bicyclic terpenes as aroma chemicals, preferably as fragrances. The invention also relates to terpene C₁-C₈-alkyl ether compositions and novel C₁-C₈-alkyl ethers of mono- and bicyclic terpenes. The present invention also relates to a method for preparing C₁-C₈-alkyl ethers of mono- and bicyclic terpenes by electrolysis of mono- or polyunsaturated, non-aromatic, mono- or bicyclic terpene hydrocarbons.

### FIELD OF INVENTION

The invention relates to the use of the terpene C₁-C₈-alkyl ether compositions and the novel C₁-C₈-alkyl ethers of mono- and bicyclic terpenes as fragrances. The invention also relates to terpene C₁-C₈-alkyl ether compositions and novel C₁-C₈-alkyl ethers of mono- and bicyclic terpenes. The present invention also relates to a method for preparing C₁-C₈-alkyl ethers of mono- and bicyclic terpenes by electrolysis of mono- or polyunsaturated, non-aromatic, mono- or bicyclic terpene hydrocarbons.

### BACKGROUND

Aroma chemicals, especially fragrances, are of great interest, especially in the field of cosmetics and cleaning and laundry compositions. Fragrances of natural origin are mostly expensive, often limited in their available amount and, on account of fluctuations in environmental conditions, are also subject to variations in their content, purity etc. To circumvent these undesirable factors, it is therefore of great interest to create synthetic substances which have organoleptic properties that resemble more expensive natural fragrances or which have novel and interesting organoleptic profiles.

Despite a large number of already existing synthetic aroma chemicals (fragrances and flavorings), there is a constant need for new components in order to be able to satisfy the multitude of properties desired for extremely diverse areas of application. These include, firstly, the organoleptic properties, i.e. the compounds should have advantageous odiferous (olfactory) or gustatory properties. Furthermore, aroma chemicals should also have additional positive secondary properties, such as e.g. an efficient preparation method, the possibility of providing better sensory profiles as a result of synergistic effects with other fragrances, a higher stability under certain application conditions, a higher extendability, a better higher substantivity, etc.

There is an increased need for aroma chemicals which can impart a sensory impression, especially a woody/fruity/sweet/dried fruit/floral/orris/powdery/foodlike, odiferous impression to a composition. Such properties are of special interest for compositions such as for example body care compositions, hygiene articles, cleaning compositions, textile detergent compositions and compositions for scent dispensers. Of special interest are aroma chemicals, which can impart one or more distinct sensory impressions to a composition, thereby contributing to a rich and interesting sensory profile, especially an olfactory profile of the composition. In addition, especially regarding aroma chemicals which can impart olfactory impression, the substantivity as well as the tenacity are of special interest in order to obtain a long-lasting odiferous impression in the composition as well as to the surface with which the composition is treated.

However, since even small changes in chemical structure bring about massive changes in the sensory properties such as odor and also flavor, the targeted search for substances with certain sensory properties such as a certain odor is extremely difficult. The search for new aroma chemicals is therefore in most cases difficult and laborious without knowing whether a substance with the desired odor and/or flavor will even actually be found.

It is an object of the presently claimed invention to provide new aroma chemicals. These should preferably have pleasant organoleptic properties.

It is a further object of the presently claimed invention to provide substances which can be used as an aroma chemical in compositions, in particular odor-intensive substances having a pleasant odor are sought. Furthermore, they should be combinable with other aroma chemicals, allowing the creation of novel advantageous sensory profiles. In addition, these aroma chemicals should be obtainable from readily available starting materials, allowing their fast and economic manufacturing.

This object is achieved by the provision of the novel compounds derived from terpenes by electrolysis using mono-, di- or tri-unsaturated, non-aromatic, mono- or bicyclic terpene hydrocarbon having 10 to 15 carbon atoms.

### SUMMARY OF THE INVENTION

A first aspect of the presently claimed invention relates to a method of preparing the compounds which are C₁-C₈-alkyl ethers of mono- and bicyclic terpenes.

Thus, the presently claimed invention relates to the use of compounds of general formula (I)

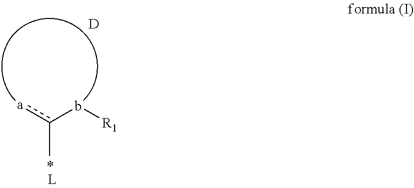

where
L is selected from the group consisting of

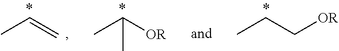

and the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—;
CH2-C(CH3)(OCH3)-CH(OCH3)-; —C═C(CH3)-CH(OCH3)-; —C═C(—CH2OCH3)-CH═; —CH2-C(O)—CH(OCH3)-; —CH2-C(CH2-O—CH3)=CH—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups
as an aroma chemical, preferably as a fragrance.

In a further aspect the presently claimed invention relates to a compound of general formula (I) as indicated above
wherein
L is selected from the group consisting of

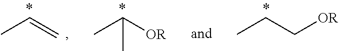

and the point of attachment of L is indicated by *;
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula (I) has 1, 2 or 3 —OR groups.

A further aspect of the presently claimed invention relates to a compound of of the general formula (II):

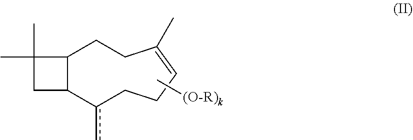

where . . . is a C—C single bond or a C═C double bond,
k is 1, 2 or 3;
R is unsubstituted, linear or branched C1-C8-alkyl and preferably unsubstituted, linear C1-C4-alkyl and the OR groups are bonded to any carbon atoms which are not part of a C═C double bond and its use to impart an aroma impression to a composition.

A yet another aspect of the presently claimed invention relates to the use of compounds of general formula (II) as an aroma chemical, preferably as a fragrance.

A further aspect of the presently claimed invention relates to a method of imparting an aroma impression to a composition comprising at least the step of adding a compound of general formula (I) or (II) as described above and below herein in a composition.

A yet further aspect of the presently claimed invention relates to a composition comprising the compounds of the presently claimed invention and (i) at least one additional aroma chemical different from the compounds of the presently claimed invention, or (ii) at least one non-aroma chemical carrier, or (iii) a mixture of (i) and (ii).

Another aspect of the presently claimed invention relates to the use of a compound of the presently claimed invention for modifying the aroma character of an aroma chemical composition.

Further aspect of the presently claimed invention relates to a method of boosting the aroma of a composition. Said method comprises the step of mixing the compound of the presently claimed invention with other ingredients such as, e.g., at least one other aroma chemical and/or at least one non-aroma chemical carrier so as to obtain the aroma chemical composition.

Yet another aspect of the presently claimed invention relates to a method of modifying the aroma of chemical composition. Said method comprises the step of incorporating the compound of the presently claimed invention into an aroma chemical composition so as to obtain an aroma-modified aroma chemical composition.

The compounds of the presently claimed invention and aroma chemical compositions thereof possess advantageous organoleptic properties, in particular a pleasant aroma impression.

Therefore, they can be favorably used as ingredients in perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions, crop protection compositions and other compositions.

The pleasant aroma impression, low volatility and excellent solubility make the compounds of the presently claimed invention a suitable ingredient in compositions where a pleasing aroma is desirable. By virtue of their physical properties, the compounds of the presently claimed invention are well combinable with other aroma chemicals and customary ingredients in perfume compositions. This allows, e.g., the creation of aroma compositions, in particular perfume compositions having novel advantageous sensory profiles.

Furthermore, the compounds of the presently claimed invention can be produced in good yields and purities by a simple synthesis starting from readily available starting materials.

Thus, the compounds of the presently claimed invention can be produced in large scales and in a simple and cost-efficient manner.

### DETAILED DESCRIPTION OF THE INVENTION

The following detailed description is merely exemplary in nature and is not intended to limit the presently claimed invention or the application and uses of the presently claimed invention.

Furthermore, there is no intention to be bound by any theory presented in the preceding technical field, background, summary or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or AA), BB) and CC) or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” or “preferred embodiment” means that a particular feature, structure or characteristic described in connection with the embodiment is included in at least one embodiment of the presently claimed invention. Thus, appearances of the phrases “in one embodiment” or “In a preferred embodiment” or “in a preferred embodiment” in various places throughout this specification are not necessarily all referring to the same embodiment but may refer. Furthermore, the features, structures or characteristics may be combined in any suitable manner, as would be apparent to a person skilled in the art from this disclosure, in one or more embodiments.

Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments are used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

### Definitions

In the context of the present invention, the term “aroma” refers to a sensory property and comprises an odor and/or a flavor.

The term “aroma chemical” denotes a substance which is used to obtain a sensory or organoleptic (used interchangeably herein) impression and comprises its use to obtain an olfactory and/or a flavor impression. The term “olfactory impression” or “note” (used interchangeably here) denotes an odor impression without any positive or negative judgement, while the term “scent impression” or “fragrance impression” or “aroma impression” (used interchangeably herein) as used herein is connected to an odor impression which is generally felt as pleasant. Thus a “fragrance” or “scent” denotes an aroma chemical, which predominately induces a pleasant odor impression. A flavor denotes an aroma chemical, which induces a taste impression.

The term “aroma composition”, as used herein, refers to a composition which induces an aroma. The term aroma composition comprises “odor composition” and/or “flavor composition”. An odor composition being a composition, which predominately induces an odor impression, a flavor composition being a composition, which predominantly induces a taste impression.

The term odor composition comprises “fragrance composition” or “scent composition” (used interchangeably herein), which predominately induce an odor impression which is generally felt as pleasant.

The general hedonistic expressions “advantageous sensory properties” or “advantageous organoleptic properties” describe the niceness and conciseness of an organoleptic impression conveyed by an aroma chemical. “Niceness” and “conciseness” are terms which are familiar to the person skilled in the art, such as a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel—brings about a reproducibly identical reminder of something specific. For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”. However, the odor of a typically tart apple can also be concise. If both reactions arise upon smelling the substance, in the example thus a nice and concise apple odor, then this substance has particularly advantageous sensory properties.

The expressions “combination of”, “in combination with” or “combined with” when used herein referring to the compositions, methods or the use of two compounds, take account of the fact that the two compounds do not need to be used in the form of a physical mixture of said compounds but can be used (e.g., added) separately. Where the compounds are used separately, they can be used (e.g. added) sequentially (i.e. one after the other) in any order, or concurrently (i.e. basically at the same time).

The term “boosting”, or “boost” is used herein to describe the effect of enhancing and/or modifying the aroma of an aroma chemical or of a composition. The term “enhancing” comprises an improvement of the niceness and/or conciseness of an aroma and/or an improvement of the intensity. The term “modifying” comprises the change of an aroma profile.

The intensity can be determined via a threshold value determination. A threshold value of an odor is the concentration of a substance in the relevant gas space at which an odor impression can just still be perceived by a representative test panel, although it no longer has to be defined.

Booster effects are particularly desired in fragrance composition when top-note-characterized applications are required, in which the odor is to be conveyed particularly quickly and intensively, for example in deodorants, air fresheners or in the taste sector in chewing gums.

The terms “the invention relates to” and “the invention is directed to” are used synonymously throughout the invention.

The terms “compound” and “substance” are used synonymously throughout the invention. The term “compound” means compounds of formula (I) or compounds of formula (II). The term “compound” also encompasses mixtures.

The term “tenacity” describes the evaporation behavior over time of an aroma chemical. The tenacity can for example be determined by applying the aroma chemical to a test strip, and by subsequent olfactory evaluation of the odor impression of the test strip. For aroma chemicals with high tenacity the time span after which the panel can still identify an aroma impression is long.

The term “substantivity” describes the interaction of an aroma chemical with a surface, such as for example the skin or a textile, especially after subsequent treatment of the surface, such as for example washing. The substantivity can for example be determined by washing a textile with a textile detergent composition comprising the aroma chemical and subsequent olfactory evaluation of the textile directly after washing (wet textile) as well as evaluation of the dry textile after prolonged storage.

The term “stability” describes the behavior of an aroma chemical upon contact with oxygen, light and/or other substances. An aroma chemical with high stability maintains its aroma profile over a long period in time, preferably in a large variety of compositions and under various storage conditions.

In order to impart a long-lasting aroma impression to a composition or to a surface treated with a composition, the tenacity, the substantivity as well as the stability of the aroma chemical in the compositions should preferably be high.

Unless specified otherwise herein, a “compound” described herein relates to the compounds defined by the general formula (I) or formula (II).

Compound:

In an embodiment of the present invention, the compound of formula (I) is

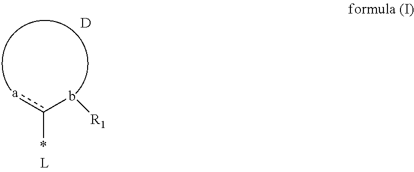

where

L is selected from the group consisting of

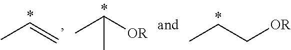

the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups.

In an embodiment of the present invention, R is methyl or ethyl.

In yet another embodiment of the present invention, R₁ is —OR.

In an embodiment of the present invention, L is

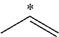

In an embodiment of the present invention, L is

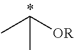

In yet another embodiment of the present invention, L is

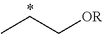

In an embodiment of the present invention, the compound of formula (I) is selected from the list of compounds in the below table C

TABLE C

| L | carbon chain D | R |
|---|---|---|
| 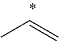 | —CH₂—C(CH₃) OR—CH (OR)— | Methyl or Ethyl |
| 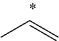 | —CH₂—C(CH₃) OR—CH═ | Methyl or Ethyl |
| 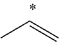 | —CH₂—C(CH₃) ═CH— | Methyl or Ethyl |
| 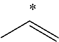 | —CH₂—CH(CH₃)—C(OR)═, | Methyl or Ethyl |
| 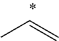 | —CH₂—C(CH₂—O— R) ═CH— | Methyl or Ethyl |
| 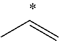 | —CH₂—C(OR)₂—CH(CH₃)— | Methyl or Ethyl |
| 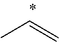 | —C(CH₃) (CH(OR)₂)—CH₂ | Methyl or Ethyl |
| 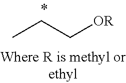 | —CH═C(CH₃)—CH(OR)— | Methyl or Ethyl |
| 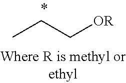 | —CH═C(CH₃)—CH(OR)— | Methyl or Ethyl |
| 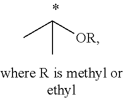 | ═CH—C(CH₃) (OR)—CH₂— | Methyl or Ethyl |
| 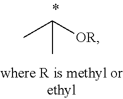 | ═CH—C(OR)(CH₂OR)—CH₂— | Methyl or Ethyl |
| 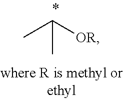 | ═CH—C(═O)—CH₂— | |

In yet another embodiment of the present invention, the compound of formula (I) is selected from the group consisting of:
1-ethoxymethyl-4-(1-ethoxy-1-methylethyl)cyclohexene;
2-ethoxy-4-(1-ethoxy-1-methylethyl)-1-methylenecyclohexane;
4-isopropenyl-1,2-diethoxy-1-methylcyclohexane;
4-isopropenyl-1,1-diethoxy-2-methylcyclohexane;
6-ethoxy-4-(1-ethoxy-1-methylethyl)-1-methylcyclohexene;
6-ethoxy-4-(2-ethoxy-1-methylethyl)-1-methylcyclohexene;
6-ethoxy-4-(1-ethoxymethylvinyl)-1-methylcyclohexene;
5-ethoxy-1-(2-ethoxy-1-methylethyl)-4-methylcyclohexa-1,3-diene;
1-ethoxymethyl-4-(1-ethoxy-1-methylethyl)benzene;
3-ethoxy-6-(1-ethoxy-1-methylethyl)-3-methylcyclohexene;
3-ethoxy-3-ethoxymethyl-6-(1-ethoxy-1-methylethyl)cyclohexene;
4-(1-ethoxy-1-methylethyl)cyclohex-2-en-1-one;
4-isopropenyl-1,2-dimethoxy-1-methylcyclohexane;
4-isopropenyl-1,1-dimethoxy-2-methylcyclohexane;
6-methoxy-4-(2-methoxy-1-methylethyl)-1-methylcyclohexene;
6-methoxy-4-(1-methoxymethylvinyl)-1-methylcyclohexene;
5-ethoxy-1-(2-methoxy-1-methylethyl)-4-methylcyclohexa-1,3-diene;
3-methoxy-6-(1-methoxy-1-methylethyl)-3-methylcyclohexene;
3-methoxy-3-methoxymethyl-6-(1-ethoxy-1-methylethyl)cyclohexene;
1-ethoxy-3-isopropenyl-6-methyl-cyclohexene;
1-ethoxy-4-isopropenyl-1-methyl-cyclohexane;
3-ethoxy-4-isopropenyl-1-methyl-cyclohexene;
1-(ethoxymethyl)-4-isopropenyl-cyclohexene;
1-(diethoxymethyl)-3-isopropenyl-1-methyl-cyclopentane
1,2-diethoxy-4-isopropenyl-1-methyl-cyclohexane; and
4-(1-methoxy-1-methylethyl)cyclohex-2-en-1-one.

In yet another embodiment of the present invention, the compound of the general formula (II) is,

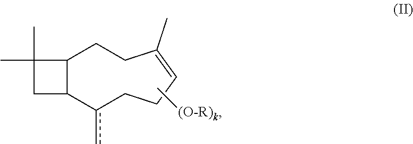

where [symbol] is a C—C single bond or a C═C double bond,
k is 1, 2 or 3;
R is unsubstituted, linear or branched C₁-C₈-alkyl and preferably unsubstituted, linear C₁-C₄-alkyl and the OR groups are bonded to any carbon atoms which are not part of a C═C double bond.

In yet another embodiment of the present invention, the compound of formula II, wherein R is methyl or ethyl.

In a further embodiment of the present invention, the compound of the general formula (II) is selected from the group consisting of
3-ethoxy-4,11,11-trimethyl-8-methylenebicyclo[7,2,0]undec-4-ene;
6-ethoxy-4,11,11-trimethyl-8-methylenebicyclo[7,2,0]undec-4-ene;
7-ethoxy-4,11,11-trimethyl-8-methylenebicyclo[7,2,0]undec-4-ene;
9-ethoxy-4,11,11-trimethyl-8-methylenebicyclo[7,2,0]undec-4-ene;
4-ethoxymethyl-11,11-dimethyl-8-methylenebicyclo[7,2,0]undec-4-ene;
6,8-diethoxy-4,8,11,11-tetramethylbicyclo[7,2,0]undec-4-ene;
8-(diethoxymethyl)-6-ethoxy-4,11,11-trimethylbicyclo[7,2,0]undec-4-ene;
3-methoxy-4,11,11-trimethyl-8-methylenebicyclo[7,2,0]undec-4-ene;
6-methoxy-4,11,11-trimethyl-8-methylenebicyclo[7,2,0]undec-4-ene;
7-methoxy-4,11,11-trimethyl-8-methylenebicyclo[7,2,0]undec-4-ene;
9-methoxy-4,11,11-trimethyl-8-methylenebicyclo[7,2,0]undec-4-ene;
4-methoxymethyl-11,11-dimethyl-8-methylenebicyclo[7,2,0]undec-4-ene;
6,8-dimethoxy-4,8,11,11-tetramethylbicyclo[7,2,0]undec-4-ene; and
8-(dimethoxymethyl)-6-methoxy-4,11,11-trimethylbicyclo[7,2,0]undec-4-ene.

Use:

One embodiment of the presently claimed invention is directed to the use of compounds of formula (I)

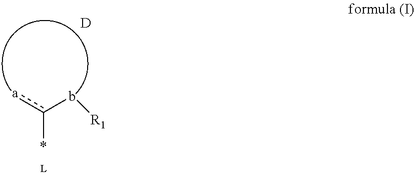

wherein
L is selected from the group consisting of

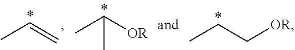

the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—;
CH2-C(CH3)(OCH3)-CH(OCH3)-; —C═C(CH3)-CH(OCH3)-; —C═C(—CH2OCH3)-CH═; —
CH2-C(O)—CH(OCH3)-; —CH2-C(CH2-O—CH3)=CH—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups,

as an aroma chemical, preferably as a fragrance.

Another embodiment of the presently claimed invention is directed to the use of compounds of formula (I), wherein the compound of formula (I) is selected from the list in the below Table U.

TABLE U

| L | carbon chain D | R |
|---|---|---|
| 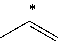 | —CH₂—C(CH₃) OR—CH(OR)— | Methyl or Ethyl |
| 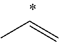 | —CH₂—C(CH₃) OR—CH═ | Methyl or Ethyl |
| 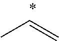 | —CH₂—C(CH₃) ═CH— | Methyl or Ethyl |
| 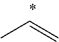 | —CH₂—CH(CH₃)—C(OR)═, | Methyl or Ethyl |
| 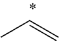 | —CH₂—C(CH₂—O—R) ═CH— | Methyl or Ethyl |
| 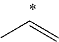 | —CH₂—C(OR)₂—CH(CH₃)— | Methyl or Ethyl |
| 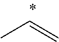 | —C(CH₃) (CH(OR)₂)—CH₂ | Methyl or Ethyl |
| 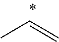 | —CH₂—C(CH₃)(OCH₃)—CH(OCH₃)— | |
| 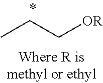 | —CH═C(CH₃)—CH(OR)— | Methyl or Ethyl |
| 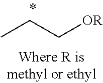 | —CH═C(CH₃)—CH(OR)— | Methyl or Ethyl |
| 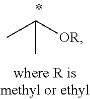 | ═CH—C(CH₃) (OR)—CH₂— | Methyl or Ethyl |
| 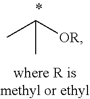 | ═CH—C(OR)(CH₂OR)—CH₂— | Methyl or Ethyl |
| 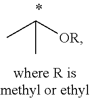 | ═CH—C(═O)—CH₂— | |
| 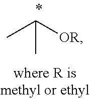 | —C═C(CH₃)—CH(OCH₃)— | |
| 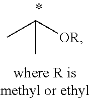 | —C═C(—CH₂OCH₃)—CH═ | |
| 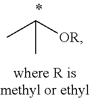 | —CH₂—C(O)—CH(OCH₃)— | |
| 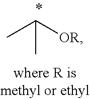 | —CH₂—C(CH₂—O—CH₃)═CH— | |

In a further embodiment of the presently claimed invention is directed to the use of compounds of formula (I) to boost the aroma of a composition.

Further embodiment of the presently claimed invention is directed to the use of compounds of formula (II) as an aroma chemical, preferably as a fragrance.

In an embodiment of the presently claimed invention, the compounds of the presently claimed invention (compound of formula (I) or formula (II)) or an aroma chemical composition comprising said compound is used as a fragrance.

In particular, the compound of the presently claimed invention is used to impart a note that is selected from the group consisting of herbal, earthy, fresh carrot like, fresh, green, floral, spicy, juniper like, sweetish, fruity, anise like, liquorice like, cinnamon like, clove like, coriander like, parsley like, dill like, tart, woody, minty, caraway like, menthol like, citrus like, resinous like notes or a combination of two or more notes.

In particular the compound of the presently claimed invention is used to impart a note that is selected from the group consisting of, pine like, aspects of resin, needle leaved forest, Fennel like, Dill like, sweaty, pungent, reminiscent of canned pickles, fatty, floral, citric, woody or a combination of two or more notes.

Suitable compositions are for example compositions used in personal care, in home care, in industrial applications as well as compositions used in other applications, such as pharmaceutical compositions or crop protection compositions.

Preferably, the compound of the presently claimed invention is used in a composition selected from the group consisting of perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions. The compound of the presently claimed invention is used as an aroma chemical, preferably as a fragrance, in the above compositions.

In particular, the compounds of formula (I) or (II) of the presently claimed invention are used to impart a note that is selected the group consisting of dried fruits, florals, sweets, orris and powdery notes; The compounds for formula (I) and/or (II) are preferably are used to impart an aroma impression that is reminiscent of sweets, florals, fruity elements to a composition.

Details to the above-listed compositions are given below.

Similarly, the compound of formula (I) and formula (II) of the presently claimed invention can improve the sensory profiles of chemical compositions as a result of synergistic effects with other aroma chemicals (e.g., other fragrances) comprised in the compositions, which means that the compound can provide a booster effect for said other aroma chemicals. The compound is therefore suitable as a booster for other aroma chemicals.

Accordingly, the presently claimed invention also relates to the use of the compounds of formula (I) or (II) of the presently claimed invention for modifying the aroma character (e.g., the scent character) of an aromatized (e.g., fragranced) composition; and specifically, to the use as a booster for other aroma chemicals.

Booster effect of a substance means that the substance enhances and intensifies in aroma chemical formulations (such as, e.g., perfumery formulations) the overall sensory (e.g., olfactory) impression of the formulation. Booster effects are particularly desired when top-note-characterized applications are required, in which the odor impression is to be conveyed particularly quickly and intensively, for example in deodorants, air fresheners or in the taste sector in chewing gums.

To achieve such a booster effect, the compound of the presently claimed invention can be used, for example, in an amount of 0.001 wt. % to 10 wt. % (weight-%), such as in an amount of 0.01 wt. % to 2 wt. %, preferably from 0.05 wt. % to 1 wt. %, in particular in an amount of from 0.1 wt. % to 0.5 wt. %, based on the total weight of the resulting aroma chemical composition.

Furthermore, the compounds of formula (I) or (II) can have further positive effects on the composition in which it is used. For example, the compound can enhance the overall performance of the composition into which it is incorporated, such as the stability, e.g. the formulation stability, the extendibility or the staying power of the composition.

Process/Method

In an embodiment of the presently claimed invention, the process for preparing the compound of formula (I) and/or (II) comprises subjecting a mono-, di- or tri-unsaturated, non-aromatic, mono- or bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

### Definitions

In the context of the definition of substituents and groups, the prefix Cₙ-Cₘ specifies the number of carbon atoms which the respective substituent or the respective group can have.

Here and hereinafter, alkyl is a linear or branched, saturated hydrocarbon residue. For instance, by way of example, C₁-C₄-alkyl is methyl, ethyl, n-propyl, isopropyl, n-butyl, 2-butyl, 2-methylpropyl (=isobutyl) or 2-methylpropan-2-yl (=tert-butyl). Accordingly, C₁-C₈-alkyl is a linear or branched alkyl having 1 to 8 carbon atoms, for example, C₁-C₄-alkyl as illustrated above, or is n-pentyl, n-hexyl, n-heptyl, n-octyl and the respective isomers.

Fluoro-C₁-C₄-alkyl is a C₁-C₄-alkyl, as defined above, in which at least one or, in particular, all hydrogen atoms have been replaced by fluorine. Examples of fluoroalkyl are particularly trifluoromethyl, pentafluoroethyl, heptafluoropropyl and heptafluoroisopropyl. Fluoro-C₁-C₄-alkyl is especially trifluoromethyl.

C₁-C₈-hydroxyalkyl is a C₁-C₈-alkyl, as defined above, in which one hydrogen atom has been replaced by OH. Hydroxyalkyl is a C₂-C₄-hydroxyalkyl such as 2-hydroxyethyl, 3-hydroxypropyl, 2-hydroxypropyl, 1-methyl-2-hydroxyethyl, 2-hydroxybutyl, 1-methylhydroxypropyl and 4-hydroxybutyl.

Aryl is an aromatic hydrocarbon residue such as phenyl, which has optionally been monosubstituted or polysubstituted by C₁-C₄-alkyl or halogen. Examples are phenyl, tolyl, p-chlorophenyl, p-fluorophenyl and pentafluorophenyl.

The term “electrolyte” refers here and hereinafter to a liquid composition which comprises the solvent in which the electrolysis is carried out, the conductive salt and the terpene hydrocarbon used.

In a first preferred embodiment, the terpene hydrocarbon has a menthene or menthadiene skeleton. Such compounds may be described by the formula (III):

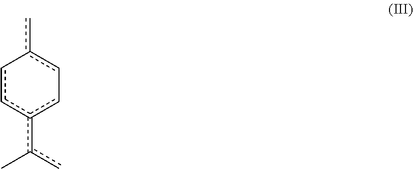

In formula (III), the variables are defined as follows:

[symbol] is a C—C single bond or a C═C double bond, with the proviso that one or two non-adjacent [symbol] bonds are a C═C double bond.

The compounds of formula (III) have one or more asymmetric carbon atoms and can therefore form enantiomers and diastereomers. In the method according to the invention they may be used as pure enantiomers or pure diastereomers, as mixtures of diastereomers or mixtures of enantiomers, e.g. as non-racemic mixtures of enantiomers or as racemic mixtures of enantiomers.

Examples of compounds having a menthene or menthadiene skeleton are limonene (formula III-a), α-phellandrene (formula III-b), β-phellandrene (formula III-c), α-terpinene (formula III-d), β-terpinene (formula III-e), γ-terpinene (formula III-f) and terpinolene (formula III-g). Of course, mixtures of formula (III) can also be used:

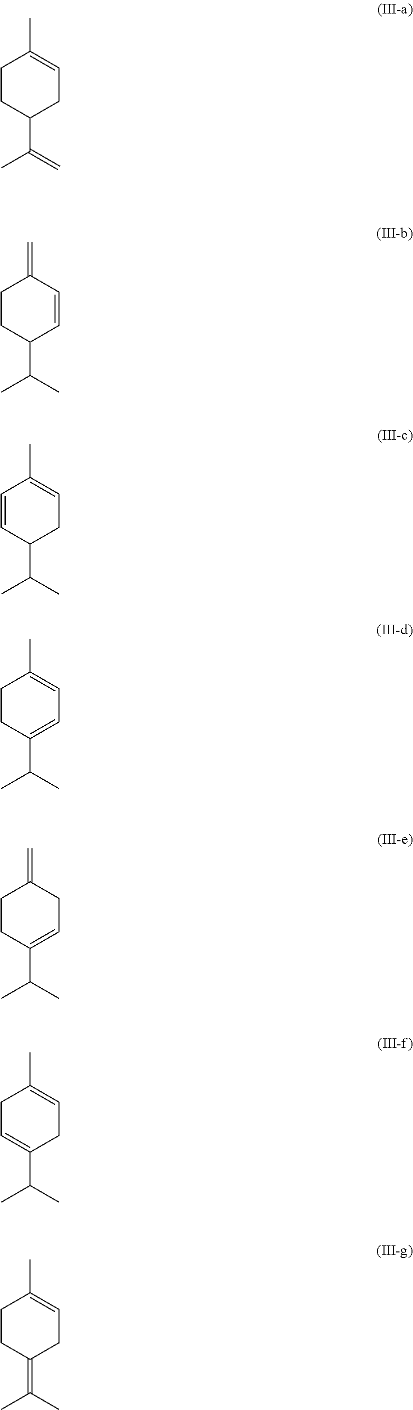

The compounds of the formulae (III-a), (III-b), (III-c) have at least one asymmetric carbon atom and may therefore be used in the method according to the invention as pure enantiomers or pure diastereomers, as mixtures of diastereomers or as mixtures of enantiomers, e.g. as non-racemic mixtures of enantiomers or as a racemic mixture.

In an embodiment of the present invention, a process for preparing a compound of formula (I), comprising subjecting mono-, di- or tri-unsaturated, non-aromatic, monocyclic terpene hydrocarbon to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

In a further embodiment of the present invention for the monocyclic terpene is selected from the group consisting of limonene, α-phellandrene, β-phellandrene, α-terpinene, β-terpinene, γ-terpinene, terpinolene.

In a second preferred embodiment, the terpene hydrocarbon is a bicyclic terpene hydrocarbon, in particular a mono- or di-unsaturated bicyclic terpene hydrocarbon, especially a mono- or di-unsaturated bicyclic terpene hydrocarbon having 10 or 15 carbon atoms. Examples thereof are in particular α-pinene (2,6,6-trimethylbicyclo[3.1.1]hept-2-ene), β-pinene (6,6-dimethyl-2-methylenebicyclo[3.1.1]heptane), camphene (2,2-dimethyl-3-methylenebicyclo[2.2.1]heptane), 3-carene (3,7,7-trimethylbicyclo[4.1.0]hept-3-ene) and β-caryophyllene (compound of formula (IV)).

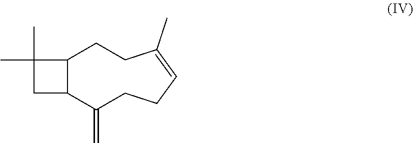

The electrolysis according to the invention is carried out in an electrolyte comprising a C₁-C₈-alkanol as main constituent. A further constituent part of the electrolyte is the conductive salt and the terpene hydrocarbon.

In an embodiment of the present invention, the process for preparing the compound of formula (II), comprising subjecting a mono-, di- or tri-unsaturated, non-aromatic, bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

In a further embodiment of the present invention the bicyclic terpene hydrocarbon is selected from the group consisting of α-pinene, β-pinene, camphene, 3-carene, β-caryophyllene The concentration of the alkanol in the electrolyte is in the range of 60 to 99% by weight, particularly in the range of 70 to 98% by weight, and especially in the range of 80 to 95% by weight, based on the total mass of the electrolyte.

The mono- or polyunsaturated terpene hydrocarbon is used typically in an amount wherein its concentration in the electrolyte at the start of the electrolysis—or in the quasi-stationary state in continuous electrolysis—is preferably in the range of 1 to 25% by weight, particularly in the range of 5 to 10% by weight, based on the total mass of the electrolyte.

The concentration of the conductive salt in the electrolyte can be in the range of 0.1 to 60% by weight, based on the total mass of the electrolyte, and is preferably in the range of 1 to 20% by weight, especially in the range of 3 to 12% by weight, based on the total mass of the electrolyte.

The C₁-C₈-alkanol is preferably selected from linear C₁-C₄-alkanols, particularly methanol and ethanol.

In accordance with the invention, the electrolyte comprises a conductive salt of which the anions are selected from the following groups:
organic sulfates, such as the sulfate anion (SO₄²⁻), hydrogen sulfate, C₁-C₈-alkyl sulfates, such as methylsulfate or ethylsulfate, and aryl sulfates such as phenylsulfate;
organic sulfonates, e.g. C₁-C₈-alkyl sulfonates, such as methanesulfonate or ethanesulfonate, aryl sulfonates such as phenylsulfonate or toluenesulfonate, fluoro-C₁-C₄-alkyl sulfonates such as triflate (trifluoromethanesulfonate=F₃C—SO₃⁻);
organic phosphates, for example mono- and di-C₁-C₈-alkyl phosphates, such as monoethyl phosphate, monobutyl phosphate, ethylhexyl phosphate, diethyl phosphate, dibutyl phosphate and mono- and di-C₁-C₄-fluoroalkyl phosphates, such as trifluoromethyl phosphate;
fluoroalkyl carboxylates such as trifluoroacetate;
sulfonylimides such as bis(fluoro-C₁-C₄-alkylsulfonyl)imides, e.g. triflimide (bis(trifluoromethylsulfonylimide)=(F₃C—SO₂)₂N⁻).

Preferred conductive salts are salts of anions selected from the following groups:
inorganic and organic sulfates, such as the sulfate anion (SO₄²⁻), hydrogen sulfate, C₁-C₈-alkyl sulfates, such as methylsulfate or ethylsulfate, aryl sulfates such as phenylsulfate;
sulfonates, e.g. C₁-C₈-alkyl sulfonates, such as methansulfonate or ethanesulfonate, aryl sulfonates, such as phenylsulfonate or toluenesulfonate, fluoro-C₁-C₄-alkyl sulfonates, such as triflate (trifluoromethanesulfonate=F₃C—SO₃⁻);
bis(fluoro-C₁-C₄-alkylsulfonyl)imides, such as triflimide.

In particular, the conductive salts are selected from salts of the following anions which are selected from the following groups:
C₁-C₄-alkyl sulfates, such as methylsulfate or ethylsulfate;
C₁-C₄-alkyl sulfonates, such as methanesulfonate or ethanesulfonate;
aryl sulfonates, such as phenylsulfonate or toluenesulfonate;
fluoro-C₁-C₄-alkyl sulfonates, such as triflate (trifluoromethanesulfonate=F₃C—SO₃⁻); and
bis(fluoro-C₁-C₄-alkylsulfonyl)imides, such as triflimide.

The cations of the conductive salts are preferably selected from alkali metal cations such as lithium, sodium or potassium, alkaline earth metal cations such as magnesium or calcium and quaternary ammonium ions.

Preference is given to alkali metal cations, especially sodium or potassium, and particularly quaternary ammonium ions. Quaternary ammonium ions are especially those of the formulae K-1 to K-4

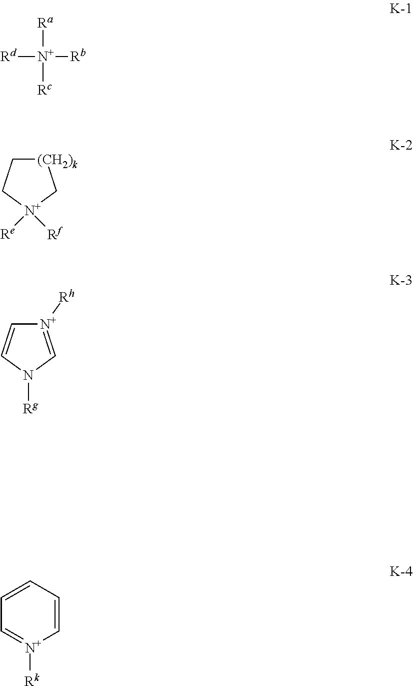

where k in formula K-2 is 1 or 2;
R^{a}, R^{b}, R^{c}, R^{d} are the same or different and are mutually independently C₁-C₈-alkyl or C₁-C₈-hydroxyalkyl;
R^{e}, R^{f} are the same or different and are mutually independently C₁-C₈-alkyl;
R^{g}, R^{h} are the same or different and are mutually independently C₁-C₈-alkyl; R^{k} is C₁-C₈-alkyl.

Examples of cations of the general formula K-1 are tetramethylammonium, tetraethylammonium, tetra-n-propylammonium, tetra-n-butylammonium and methyltri-n-butylammonium.

Examples of cations of the general formula K-2 are N,N-dimethylpyrrolidinium, N,N-diethylpyrrolidinium, N-methyl-N-propylpyrrolidinium and N-methyl-N-butylpyrrolidinium.

Examples of cations of the general formula K-3 are N,N′-dimethylimidazolium, N,N′-diethylimidazolium, N-methyl-N′-ethylimidazolium, N-methyl-N′-propylimidazolium and N-methyl-N-butylimidazolium.

Examples of cations of the general formula K-4 are N-methylpyridinium, N-ethylpyridinium, N-(n-propyl)pyridinium and N-butylpyridinium.

Examples of conductive salts are sodium methyl sulfate, tetraethylammonium sulfate, tetra-n-butylammonium sulfate, tetraethylammonium methyl sulfate, tetraethylammonium tosylate, tetra-n-butylammonium methylsulfate, methyl-tri-n-butylammonium methylsulfate (MTBS), methyltriethylammonium methylsulfate (MTES), methyl-tri-n-butylammonium methanesulfonate, methyl-tris(2-hydroxyethyl)lammonium methylsulfate, sodium methyl sulfate, N,N′-dimethylimidazolium methylsulfate, N,N′-diethylammonium ethylsulfate, N-methyl-N′-ethylimidazolium methylsulfate, N-methyl-N′-ethylimidazolium ethylsulfate, N-methyl-N′-n-butylimidazolium methylsulfate, N,N′-dimethylimidazolium tosylate, N,N′-diethylammonium tosylate, N-methyl-N′-ethylimidazolium tosylate, N-(n-butyl)-N-methylpyrrolidinium triflimide, methyl-tri-n-butylammonium triflimide, dimethylimidazolium triflimide, N-methyl-N′-ethylimidazolium triflimide and N-methyl-N′-n-butylimidazolium triflimide.

In addition to the alkanol, the unsaturated terpene hydrocarbon and the conductive salt, the electrolyte may also comprise inert solvents. Preference is given to polar aprotic solvents as inert solvents. The proportion of inert solvents generally does not exceed 40% by weight, particularly 30% by weight and especially 10% by weight. Examples of inert solvents are N,N-dialkylamides such as dimethylformamide and dimethylacetamide, N-alkyllactams such as N-methylpyrrolidone, N-methylpiperidone, alkylene carbonates such as ethylene carbonate and propylene carbonate, dialkyl ethers such as diethyl ether, tert-butyl methyl ether, tert-butyl ethyl ether, alicyclic ethers such as tetrahydrofuran, methyltetrahydrofuran and dioxane and also mixtures of these solvents.

The electrolyte generally comprises no, or only low amounts, of water. The electrolyte generally comprises less than 30% by weight, particularly less than 10% and especially less than 1% by weight of water, based on the total mass of the electrolyte.

The conversion of the unsaturated terpene hydrocarbon to the terpene C₁-C₈-alkyl ether takes place at the anode. The reaction is presumably initiated by abstraction of allylic hydrogen forming an allylic radical, followed by transfer of an electron to the anode to form an allylic cation. Hydride abstraction occurring at the anode is also feasible. The allylic cation then reacts with the C₁-C₈-alkanol to form a terpene C₁-C₈-alkyl ether.

Suitable electrode materials for the anode are in principle electrically conductive semimetals and metals which are stable under the electrolysis conditions. Noble metals such as platinum and platinum alloys have proven to be advantageous, and also particularly carbon materials, i.e. electrode materials comprising elemental carbon as main constituent.

Suitable carbon materials are, for example, graphite, boron-doped diamond, carbon felt, glassy carbon, reticulated vitreous carbon, supported carbon paper, for example in the form of a gas diffusion layer electrode. Preferred carbon materials are graphite, glassy carbon, carbon paper and boron-doped diamond.

In principle, all electrode forms known to those skilled in the art may be used as anode. These may consist wholly of the respective anode material or be a support electrode having a support which is coated with anode material. The electrodes used as anode may be configured as full-surface, reticular or lattice shaped, as foam or electrodes with gas diffusion layer (GDL electrodes).

In principle, all electrodes suitable for electrolysis of alcoholic electrolytes and known to those skilled in the art may be used as cathode. Since reduction processes take place at the cathode and the terpene hydrocarbon is oxidized at the anode, when using a heavy metal as electrode material for the cathode, for example, mercury, nickel, lead, tin or alloys thereof, the contamination of the electrolyte with this heavy metal is so low that the terpene C₁-C₈-alkyl ether composition obtained may be readily used in cosmetics or in foodstuffs. The electrode materials preferably show a low hydrogen overpotential. Electrodes suitable as cathode are, for example, those having an electrode material selected from nickel, Ni-based alloys, iron, Fe-based alloys, including steel, copper, Cu-based alloys, silver, silver, Ag-based alloys, tin, tin alloys, lead, mercury, titanium, platinum, and carbon materials such as graphite, carbon, glassy carbon, carbon felt, carbon paper, reticulated vitreous carbon and boron-doped diamond. In particular, the electrode material of the cathode is selected from nickel, Ni-based alloys, iron, Fe-based alloys, including steel, copper, Cu-based alloys, and the aforementioned carbon materials, especially graphite, glassy carbon and supported carbon paper.

In principle, all electrode forms known to those skilled in the art may be used as cathode. This may consist wholly of the respective electrode material or be a support electrode having an electrically conductive support which is coated with the electrode material.

The arrangement of anode and cathode is not limited and comprises, for example, arrangements of planar lattices and/or plates, which can also be arranged in the form of several, alternating polarized stacks, and cylindrical arrangements of cylindrically shaped meshes, lattices or tubes, which may also be arranged in the form of several, alternating polarized cylinders.

Various electrode geometries for achieving optimal space-time yields are known to those skilled in the art. Advantageous are a bipolar arrangement of several electrodes, an arrangement in which a rod-shaped anode is enclosed by a cylindrical cathode, or an arrangement in which both the cathode and the anode consist of a wire mesh and these wire meshes were placed on one another and rolled up cylindrically.

The anode and the cathode can be separated from each other by a separator. In principle, all separators customarily used in electrolysis cells are suitable as separators. The separator is typically a porous flat structure arranged between the electrodes, e.g. a lattice, mesh, woven or non-woven fabric, composed of an electrically non-conducting material which is inert under the electrolysis conditions, e.g. a plastic material, particularly a teflon material or a plastic material coated with teflon.

Any electrolysis cells known to those skilled in the art can be used for the electrolysis, such as a divided or undivided continuous-flow cell, capillary gap cell or stacked plate cell.

Undivided electrolysis cells are preferred, i.e. anode and cathode are not separated by a separator.

In particular, the electrolysis is carried out in an electrolysis cell with a bipolar electrode arrangement, in particular in a bipolar stacked plate cell. Such a stacked plate cell has several plate-shaped electrodes, which are arranged in a stack and are alternatingly polarized.

The method according to the invention can also be carried out on an industrial scale. Appropriate electrolysis cells are known to those skilled in the art. All embodiments of this invention refer not only to the laboratory scale but also to the industrial scale.

The content of the electrolysis cell can be mixed. Any mechanical stirrer known to those skilled in the art can be used for said mixing of the cell content. Preference is also given to the use of other mixing methods, such as the use of Ultra-turrax, ultrasound or jet nozzles.

By applying the electrolysis voltage to the anodes and the cathodes, electric current is conducted through the electrolyte. In order to avoid side reactions such as overoxidation and detonating gas formation, generally a current density of 200 mA/cm², frequently 150 mA/cm², particularly 100 mA/cm² and especially 60 mA/cm² is not exceeded. The current densities at which the method is carried out are generally 1 to 150 mA/cm², preferably 5 to 100 mA/cm².

Particular preference is given to carrying out the method according to the invention at current densities of between 15 and 60 mA/cm².

The amount of charge applied is generally in the range of 0.3 to 10 F per mol of unsaturated terpene hydrocarbon, particularly in the range of 1 to 8 F per mol and especially in the range of 2 to 6 F per mol of unsaturated terpene hydrocarbon.

The electrolysis can be carried out galvanostatically, i.e. at constant current, or potentiostatically, i.e. at constant clamping voltage. The electrolysis is preferably carried out galvanostatically.

The total duration of the electrolysis naturally depends on the electrolysis cell, the electrodes used and the current density. An optimal duration can be determined by those skilled in the art by routine experiments, for example, by sampling during the electrolysis.

In order to avoid a deposit on the electrodes, the polarity can be reversed in short time intervals. The polarity can be reversed at an interval of 30 seconds to 10 minutes, an interval of 30 seconds to 2 minutes being preferred. It is appropriate in this case that the anode and cathode consist of the same material.

The electrolysis is carried out according to the method according to the invention generally at a temperature in a range of 0 to 80° C., preferably 15 to 60° C., particularly 20 to 50° C.

In the method according to the invention, the electrolysis is carried out generally at a pressure of at most 1000 kPa, particularly at most 500 kPa, especially at most 300 kPa, e.g. in the range of 80 to 1000 kPa, particularly 85 to 500 kPa, especially 90 to 300 kPa. Particular preference is given to carrying out the method according to the invention at a pressure in the range of atmospheric pressure (101±10 kPa).

In a particularly preferred embodiment, the method according to the invention is carried out at a temperature in the range of 20 to 50° C. and in the range of atmospheric pressure (101±20 kPa).

The method according to the invention leads generally to a mixture of various terpene C₁-C₈-alkyl ethers, wherein the exact composition is naturally determined by the starting material but also by the electrolysis conditions.

The terpene C₁-C₈-alkyl ether composition obtainable according to the invention comprise at least one di- or tri-C₁-C₈-alkyl ether and/or at least one keto-C₁-C₈-alkyl ether of a monocyclic terpene, if a terpene having a menthene or menthadiene skeleton, e.g. a compound of the general formula III, particularly of the general formula III-a to III-g, or a pinene is used as starting material. Terpene C₁-C₈-alkyl ether compositions are frequently obtainable in this manner comprising at least one mono-, di- or tri-unsaturated di- or tri-C₁-C₈-alkyl ether and/or at least one keto-C₁-C₈-alkyl ether of a monocyclic terpene. In particular, such terpene C₁-C₈-alkyl ether compositions comprise at least one mono-, di- or tri-unsaturated di- or tri-C₁-C₈-alkyl ether of a terpene having a menthene or menthadiene skeleton and optionally one or more keto-C₁-C₈-alkyl ethers of a monocyclic terpene. Specifically, such terpene C₁-C₈-alkyl ether compositions comprise at least one compound of the general formula (I), as defined above.

The term terpene C₁-C₈-alkyl ether comprises particularly terpene C₁-C₄-alkyl ethers and especially terpene methyl ethers and terpene ethyl ethers, independently of whether they are monocyclic or bicyclic, mono-, di- or tri-unsaturated mono-, di- or tri-C₁-C₈-alkyl ethers.

In an embodiment of the present invention, in the method for preparing C₁-C₈-alkyl ethers of mono- and bicyclic terpenes the at least one of the features a) to l) is included:
a) the electrolyte comprises less than 30% by weight of water, based on the total mass of the electrolyte;
b) the concentration of the alkanol in the electrolyte is in the range of 60 to 98% by weight, based on the total mass of the electrolyte;
c) the concentration of the terpene in the electrolyte is in the range of 1 to 25% by weight, based on the total mass of the electrolyte;
d) the electrolyte comprises at least one conductive salt at a concentration in the range of 1 to 20% by weight, based on the total mass of the electrolyte;
e) the conductive salt is selected from salts of which the anions are selected from C₁-C₈-alkyl sulfates, C1-C8-alkyl sulfonates, aryl sulfonates and bis(fluoro-C₁-C₄-alkylsulfonyl)imides;
f) the anode material is a carbon material;
g) the electrolysis is carried out in an undivided electrolysis cell;
h) the electrolysis is carried out galvanostatically;
i) the electrolysis is carried out in an electrolysis cell with a bipolar electrode arrangement;
j) the electrolysis is carried out in a bipolar stacked plate cell;
k) the electrolysis is carried out with a quantity of electricity of 0.3 to 10 F per mol of terpene;
l) the electrolysis is carried out with a current density in the range of 5 to 80 mA/m2.

Composition:

In an embodiment, the presently claimed invention relates to a composition comprising the compounds of formula (I) or (II) and:
(i) at least one compound other than compounds for formula (I) or (II), or
(ii) at least one non-aroma chemical carrier, or
(iii) both of (i) and (ii).

Preferably, the composition is an aroma composition.

The non-aroma chemical carrier in the composition of the invention can be selected from surfactants, oil components and solvents.

The aroma chemical (i) is different from the at least one compounds according to the invention.

By virtue of its physical properties, the compounds of the present invention are well combinable with aroma chemicals (e.g., fragrances) and other customary ingredients in aromatized (e.g., fragranced), perfume compositions. This allows, e.g., the creation of aroma compositions (e.g., perfume compositions) which have novel advantageous sensory profiles.

Especially, as already explained above, the compound can provide a booster effect for aroma chemicals (such as fragrances).

Accordingly, the composition according to the invention comprises at least one compound of formula (I) or (II), as defined herein; and at least one aroma chemical that is different from the compound of formula (I) or (II) of the presently claimed invention.

The aroma chemical (i) can for example be one, preferably 2, 3, 4, 5, 6, 7, 8 or aroma chemicals, selected from the group consisting of:
geranyl acetate, alpha-hexylcinnamaldehyde, 2-phenoxyethyl isobutyrate, dihydromyrcenol, methyl dihydrojasmonate, 4,6,6,7,8,8-hexamethyl-1,3,4,6,7,8-hexahydro-cyclopenta[g]benzopyran, tetrahydrolinalool, ethyllinalool, benzyl salicylate, 2-methyl-3-(4-tert-butylphenyl)propanal, cinnamyl alcohol, 4,7-methano-3a,4,5,6,7,7a-hexahydro-5-indenyl acetate and/or 4,7-methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexene-carboxaldehyde, alpha-amylcinnamaldehyde, ethylene brassylate, (E)- and/or (Z)-3-methylcyclopentadec-5-enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate, 2,5,5-trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate and 3-[5,5,6-trimethylbicyclo[2.2.1]hept-2-yl]cyclohexan-1-ol.

In yet another preferred embodiment, the at least one aroma chemical (i) is selected from the group consisting of methyl benzoate, benzyl acetate, geranyl acetate, 2-isobutyl-4-methyltetrahydro-2H-pyran-4-ol, linalool, 2-isobutyl-4-methyltetrahydro-2H-pyran-4-ol and methyl benzoate.

In yet another preferred embodiment, the at least one aroma chemical (i) is selected from the group consisting of ethylvanillin, vanillin, 2,5-dimethyl-4-hydroxy-2H-furan-3-one (furaneol) or 3-hydroxy-2-methyl-4H-pyran-4-one (maltol).

Further aroma chemicals with which the compound of the present invention can be combined to give a composition according to the presently claimed invention can be found, e.g., in S. Arctander, Perfume and Flavor Chemicals, Vol. I and II, Montclair, N.J., 1969, self-published or K. Bauer, D. Garbe and H. Surburg, Common Fragrance and Flavor Materials, 4th Ed., Wiley-VCH, Weinheim 2001. Specifically, mention may be made of:
extracts from natural raw materials such as essential oils, concretes, absolutes, resins, resinoids, balsams, tinctures such as e.g.
ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;
individual fragrances from the group of hydrocarbons, such as e.g. 3 carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1 octen-3-ol; mixture of3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;
the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as e.g. 2-nonenenitrile; 2-undecenenitrile; 2 tridecenenitrile; 3,12-tridecadienenitrile; 3,7-dimethyl-2,6-octadienenitrile; 3,7-dimethyl-6 octenenitrile; the esters of aliphatic carboxylic acids such as e.g. (E) and (Z)-3-hexenyl formate; ethyl acetoacetate; isoamyl acetate; hexyl acetate; 3,5,5-trimethylhexyl acetate; 3 methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;
the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2 butenoates thereof;
the acyclic terpene aldehydes and ketones such as e.g. geranial; neral; citronellal; 7 hydroxy-3,7-dimethyloctanal; 7 methoxy-3,7-dimethyloctanal; 2,6,10-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpine-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methano,naphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as e.g. alpha-3,3-trimethylcyclohexylmethanol; 1 (4-isopropylcyclohexyl)ethanol; 2-methyl-4-(2,2,3-trimethyl-3-cyclopent-1-yl)butanol; 2-methyl-4-(2,2,3 trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3 trimethyl-3-cyclopent-1-yl)pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxymethoxy)cyclo-dodecane; alpha-cedrene epoxide; 3a,6,6,9a-tetramethyldodecahydronaphtho[2,1-b]furan; 3a-ethyl-6,6,9a-trimethyldodecahydro-naphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5 trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2 cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclo-hexadecen-1-one; 7-cyclohexadecen-1-one; (7/8)-cyclohexadecen-1-one; 9 cyclo-heptadecen-1-one; cyclopentadecanone; cyclohexadecanone;
the cycloaliphatic aldehydes such as e.g. 2,4-dimethyl-3-cyclohexenecarbaldehyde; 2 methyl-4-(2,2,6-trimethylcyclohexen-1-yl)-2-butenal; 4-(4-hydroxy-4-methylpentyl)-3 cyclohexene carbaldehyde; 4-(4-methyl-3-penten-1-yl)-3-cyclohexenecarbaldehyde;
the cycloaliphatic ketones such as e.g. 1-(3,3-dimethylcyclohexyl)-4-penten-1-one; 2,2 dimethyl-1-(2,4-dimethyl-3-cyclohexen-1-yl)-1-propanone; 1-(5,5-dimethyl-1 cyclo-hexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7 methanooctahydro-5 or 6-indenyl acetate;
the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexene-carboxylate; ethyl 2-methyl-1,3-dioxolane-2-acetate;
the araliphatic alcohols such as e.g. benzyl alcohol; 1-phenylethyl alcohol, 2 phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2 phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxy,benzyl alcohol; 1-(4-isopropylphenyl)ethanol;
the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;
the aromatic and araliphatic aldehydes such as e.g. benzaldehyde; phenylacetaldehyde; 3-phenylpropanal; hydratropaaldehyde; 4-methylbenzaldehyde; 4 methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3 methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone; 4-methylacetophenone; 4-methoxyacetophenone; 4-tert-butyl-2,6-dimethylaceto-phenone; 4-phenyl-2-butanone; 4-(4-hydroxyphenyl)-2-butanone; 1-(2-naphthalenyl)-ethanone; 2-benzofuranylethanone; (3-methyl-2-benzofuranyl)ethanone; benzo,phenone; 1,1,2,3,3,6-hexamethyl-5-indanyl methyl ketone; 6-tert-butyl-1,1 dimethyl-4 indanyl methyl ketone; 1-[2,3-dihydro-1,1,2,6-tetramethyl-3-(1-methylethyl)-1H-5 indenyl]ethanone; 5′,6′,7′,8′-tetrahydro-3′,5′,5′,6′,8′,8′-hexamethyl-2-acetonaphthone;
the aromatic and aliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5 tert-butylbenzene; 3,5-dinitro-2,6-dimethyl-4-tert-butylacetophenone; cinnamonitrile; 3 methyl-5-phenyl-2-pentenonitrile; 3-methyl-5-phenylpentanonitrile; methyl anthranilate; methyl-N-methylanthranilate; Schiff bases of methyl anthranilate with 7 hydroxy-3,7-dimethyloctanal, 2-methyl-3-(4-tert-butylphenyl)propanal or 2,4 dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3 isopropyl-pyrazine; 2-isobutyl-3-methoxypyrazine;
the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2 ethoxy-5-(1-propenyl)phenol; p-cresyl phenylacetate;
the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The aroma chemical (i) used in the composition are obtained from known commercial sources and procured from Germany.

In a preferred embodiment, the at least one non-aroma chemical carrier (ii) is selected from the group consisting of surfactants, oil components, antioxidants, deodorant-active agents and solvents.

In the context of the presently claimed invention, a “solvent” serves for the dilution of the compound of the present invention to be used according to the invention and/or any further component of the composition without having its own aroma.

The amount of solvent(s) is selected depending on the composition.

In yet another preferred embodiment, the solvent is selected from the group consisting of ethanol, isopropanol, diethylene glycol monoethyl ether, glycerol, propylene glycol, 1,2-butylene glycol, dipropylene glycol, triethyl citrate and isopropyl myristate.

In yet another preferred embodiment, the solvent is present in the composition in an amount of 0.01 wt. % to 99.0 wt. %, more preferably in an amount of 0.05 wt. % to 95.0 wt. %, yet more preferably in an amount of 0.1 wt. % to 80.0 wt. %, most preferably 0.1 wt. % to 70.0 wt. %, particularly in an amount of 0.1 wt. % to 60.0 wt. %, based on the total weight of the composition.

In yet another preferred embodiment of the invention, the composition comprises 0.05 wt. % to 10 wt. %, more preferably 0.1 wt. % to 5 wt. %, yet more preferably 0.2 wt. % to 3 wt. % solvent(s), based on the total weight of the composition. In yet another preferred embodiment of the invention, the composition comprises 20 wt. % to 70 wt. %, more preferably 25 wt. % to 50 wt. % of solvent(s), based on the total weight of the composition.

One embodiment of the invention is directed to a composition comprising the compound of the present invention and at least one oil component.

In a preferred embodiment, the oil components are present in an amount of 0.1 to 80 wt. %, more preferably 0.5 to 70 wt. %, yet more preferably 1 to 60 wt. %, even more preferably 1 to 50 wt. %, particularly 1 to 40 wt. %, more particularly 5 to 25 wt. % and specifically 5 to 15 wt. %, based on the total weight of the composition.

The oil components may be selected, for example, from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C18-C38 alkyl-hydroxycarboxylic acids with linear or branched C6-C22 fatty alcohols, more especially dioctyl malate, esters of linear and/or branched fatty acids with polyhydric alcohols (for example propylene glycol, dimer dial or trimer triol), triglycerides based on C6-C10 fatty acids, liquid mono-, di- and triglyceride mixtures based on C6-C18 fatty acids, esters of C6-C22 fatty alcohols and/or Guerbet alcohols with aromatic carboxylic acids, more particularly benzoic acid, esters of dicarboxylic acids with polyols containing 2 to 10 car-bon atoms and 2 to 6 hydroxyl groups, vegetable oils, branched primary alcohols, substituted cyclohexanes, linear and branched C6-C22 fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C6 to C22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons or mixtures thereof.

It is to be understood that anti-oxidants are able to inhibit or prevent the undesired changes in the compositions to be protected caused by oxygen effects and other oxidative processes.

The effect of the antioxidants consists in most cases in them acting as free-radical scavengers for the free radicals which arise during autoxidation.

In a preferred embodiment, the antioxidant is selected from the group consisting of
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=p-Alanyl-L-histidine) and derivatives thereof,
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine)
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate),
coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA),
nordihydroguaiacic acid, nordihydroguaiaretic acid, trihydroxybutyrophenone, uric acid and derivatives thereof, mannose and derivatives thereof,
superoxide dismutase,
zinc and derivatives thereof (for example ZnO, ZnSO4),
selenium and derivatives thereof (for example selenomethionine) and
stilbenes and derivatives thereof (e.g. stilbene oxide, trans-stilbene oxide).

In a preferred embodiment, the anti-oxidant is selected from the group consisting of pentaerythrityl, tetra-di-t-butyl-hydroxyhydrocinnamate, nordihydroguaiaretic acid, ferulic acid, resveratrol, propyl gallate, butylhydroxytoluene (BHT), butylhydroxyanisole (BHA), ascorbyl palmitate and tocopherol.

In yet another preferred embodiment, the compositions according to the presently claimed invention can comprise the anti-oxidant in an amount of 0.001 to 25 wt.-%, preferably 0.005 to 10 wt.-%, more preferably 0.01 to 8 wt.-%, yet more preferably 0.025 to 7 wt.-%, even more preferably 0.05 to 5 wt.-%, based on the total weight of the composition.

Deodorizing compositions (deodorants and antiperspirants) counteract, mask or eliminate body odors. Body odors are formed through the action of skin bacteria on apocrine perspiration which results in the formation of unpleasant-smelling degradation products.

One embodiment of the invention is therefore directed to a composition comprising the compound of the present invention and at least one deodorant-active agent. In a preferred embodiment, the deodorant-active agent is selected from the groups consisting of anti-perspirants, esterase inhibitors and antibacterial agents.

Suitable antiperspirant is selected from the group consisting of salts of aluminum, zirconium or zinc. Examples are aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine. Aluminum chlorohydrate, aluminum zirconium tetrachlorohydrate, aluminum zirconium pentachlorohydrate and complex compounds thereof are preferably used.

In a preferred embodiment, the anti-perspirant is selected from the group consisting of aluminum chloride, aluminum chlorohydrate, aluminum dichlorohydrate, aluminum sesquichlorohydrate, aluminum hydroxyallantoinate, aluminum chloride tartrate, aluminum zirconium trichlorohydrate, aluminum zirconium tetrachlorohydrate and aluminum zirconium pentachlorohydrate.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

In a preferred embodiment, the esterase inhibitor is selected from the group consisting of trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester and zinc glycinate.

The compositions according to the presently claimed invention can comprise the esterase inhibitor in the range of 0.01 to 20 wt.-%, preferably 0.1 to 10 wt.-% and more particularly 0.5 to 5 wt.-%, based on the total weight of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

In a preferred embodiment, the antibacterial agent is selected from the group consisting of chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides.

The composition according to the presently claimed invention comprises the antibacterial agent in the range of 0.01 to 5 wt. % and preferably 0.1 to 2 wt.-%, based on the total weight of the composition.

Due to the characteristic sensory property of the compound of the present invention and its substantivity, tenacity as well as stability, it can especially be used to provide an odor, preferably a fragrance impression to surfactant-containing compositions such as, for example, cleaners (in particular laundry care products and all-purpose cleaners). It can preferably be used to impart a long-lasting flowery and/or a marine and/or a green and/or a sweet note and/or a rubbery note and/or a nutty note and/or a woody note and/or a dusty note and/or a rooty note and/or a lemon note to a surfactant comprising composition.

The compositions according to the presently claimed invention can thus preferably comprise at least one surfactant.

In a preferred embodiment, the surfactant is selected from the group consisting of anionic, non-ionic, cationic, amphoteric and zwitterionic surfactants. In yet another preferred embodiment, the surfactant is an anionic surfactant.

The compositions according to the invention usually contain the surfactant(s), in the aggregate, in an amount of 0 to 40 wt. %, preferably 0 to 20 wt. %, more preferably 0.1 to 15 wt. %, and particularly 0.1 to 10 wt. %, based on the total weight of the composition. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one COO(−) or SO3(−) group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C8 to C18 alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalk-ylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group.

Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C12-C18 alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolysates.

One embodiment of the presently claimed invention is directed to a composition which is selected from the group consisting of perfume compositions, body care compositions, hygiene articles, cleaning compositions, textile detergent compositions, compositions for scent dispensers, foods, food supplements, pharmaceutical compositions and crop protection compositions.

Said composition is preferably an aroma chemical composition, more preferably a fragrance composition.

Suitable compositions are for example perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions and products for oral and dental hygiene, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks and deodorant creams, products of decorative cosmetics such as e.g. eye-liners, eye-shadows, nail varnishes, make-ups, lipsticks and mascara, and products for oral and dental hygiene, such as toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as e.g. cleaners for solid surfaces, can be selected from perfumed acidic, alkaline and neutral cleaners, such as e.g. floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pretreatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, soft gels, gel caps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

In a preferred embodiment, the composition further comprises at least one auxiliary agent selected from the group consisting of preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono- or polyunsaturated fatty acids, α-hydroxy acids, polyhydroxy fatty acids, liquefiers, dyes, color-protection agents, pigments, anti-corrosives, polyols, electrolytes and silicone derivatives.

One embodiment of the invention is directed to the method of preparing a composition comprising:
(i) at least one compound other than compounds for formula (I) or (II), or
(ii) at least one non-aroma chemical carrier, or both of (i) and (ii).

For example, the method can be carried out by mixing the compound of formula (I) or (II) of the presently claimed invention described herein and:
(i) at least one compound other than compounds for formula (I) or (II), or
(ii) at least one non-aroma chemical carrier, or
(iii) both of (i) and (ii).

The invention is also directed to a method for boosting the aroma impression of a composition such as a fragranced composition, wherein the method comprises incorporating the compound of the presently claimed invention described herein into a composition.

In particular, the invention is directed to a method of preparing a perfume composition, body care composition, hygiene article, cleaning composition, textile detergent composition, composition for scent dispensers, food, food supplement, pharmaceutical composition or crop protection composition, comprising including the compound of the presently claimed invention described herein in a perfume composition, body care composition, hygiene article, cleaning composition, textile detergent composition, composition for scent dispensers, food, food supplement, pharmaceutical composition or crop protection composition.

In one embodiment the invention is directed to a method for imparting a note reminiscent of sweet and/or fruity and/or floral and/or orris and/or powdery elements to a perfume composition, body care composition, hygiene article, cleaning composition, textile detergent composition, composition for scent dispensers, food, food supplement, pharmaceutical composition or crop protection composition, which comprises including a compound of the presently claimed invention in a perfume composition, body care composition, hygiene article, cleaning composition, textile detergent composition, composition for scent dispensers, food, food supplement, pharmaceutical composition or crop protection composition.

### Embodiments

In the following, there is provided a list of embodiments to further illustrate the present disclosure without intending to limit the disclosure to the specific embodiments listed below.
1. A method for preparing C₁-C₈-alkyl ethers of mono- and bicyclic terpenes, comprising subjecting a mono-, di- or tri-unsaturated, non-aromatic, mono- or bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.
2. Method according to embodiment 1, wherein the C₁-C₈-alkyl ethers of mono- and bicyclic terpenes is selected from at least one compound of the formula (I)

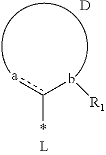

wherein
L is selected from the group consisting of

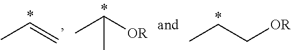

the point of attachment of L is indicated by
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—;
—CH2-C(CH3)(OCH3)-CH(OCH3)-; —C═C(CH3)-CH(OCH3)-; —C═C(—CH2OCH3)-CH═; —
CH2-C(O)—CH(OCH3)-; —CH2-C(CH2-O—CH3)=CH—,
wherein “a” and “b” denote carbon atoms and are linked via the above carbon chain
D to form a 5- or 6-membered ring,
R is unsubstituted, linear or branched C₁-C₈-alkyl, and
R₁ is H or —OR,
or formula (II)

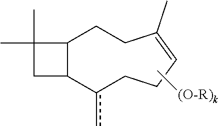

where [symbol] is a C—C single bond or a C═C double bond,
k is 1, 2 or 3;
R is unsubstituted, linear or branched C₁-C₈-alkyl and preferably unsubstituted, linear C₁-C₄-alkyl and the OR groups are bonded to any carbon atoms which are not part of a C═C double bond.
3. The method according to any of the preceding embodiments, wherein the terpene hydrocarbon has a menthene or menthadiene skeleton and is selected from limonene, α-phellandrene, β-phellandrene, α-terpinene, β-terpinene, γ-terpinene, terpinolene and mixtures thereof.
4. The method according to any of the preceding embodiments, wherein the terpene hydrocarbon is a bicyclic terpene hydrocarbon which is selected from α-pinene, β-pinene, camphene, β-carene, β-caryophyllene and mixtures thereof.
5. The method according to any of the preceding embodiments, wherein the C₁-C₈-alkanol is selected from the group consisting of linear C₁-C₄-alkanols, preferably methanol or ethanol.
6. The method according to any of the preceding embodiments having at least one of the features a) to l):
   a) the electrolyte comprises less than 30% by weight of water, based on the total mass of the electrolyte;
   b) the concentration of the alkanol in the electrolyte is in the range of 60 to 98% by weight, based on the total mass of the electrolyte;
   c) the concentration of the terpene in the electrolyte is in the range of 1 to 25% by weight, based on the total mass of the electrolyte;
   d) the electrolyte comprises at least one conductive salt at a concentration in the range of 1 to 20% by weight, based on the total mass of the electrolyte;
   e) the conductive salt is selected from salts of which the anions are selected from C₁-C₈-alkyl sulfates, C1-C8-alkyl sulfonates, aryl sulfonates and bis(fluoro-C₁-C₄-alkylsulfonyl)imides;
   f) the anode material is a carbon material;
   g) the electrolysis is carried out in an undivided electrolysis cell;
   h) the electrolysis is carried out galvanostatically;
   i) the electrolysis is carried out in an electrolysis cell with a bipolar electrode arrangement;
   j) the electrolysis is carried out in a bipolar stacked plate cell;
   k) the electrolysis is carried out with a quantity of electricity of 0.3 to 10 F per mol of terpene;
   l) the electrolysis is carried out with a current density in the range of 5 to 80 mA/m2.
7. Use of a compound of the general formula (I), according to embodiment 2

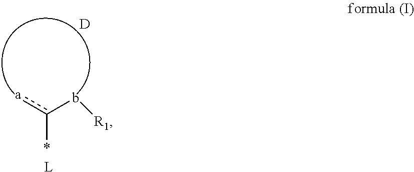

wherein
L is selected from the group consisting of

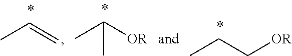

the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—;
—CH2-C(CH3)(OCH3)-CH(OCH3)-; —C═C(CH3)-CH(OCH3)-; —C═C(—CH2OCH3)-CH═; —
CH2-C(O)—CH(OCH3)-; —CH2-C(CH2-O—CH3)=CH—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups,
as an aroma chemical, preferably as a fragrance.
8. The use according to embodiment 7, wherein R is methyl or ethyl.
9. Method of imparting an aroma impression, preferably a fragrance impression, to a composition comprising at least the step of adding a compound as described in embodiment 7 or 8 to a composition.
10. The use or method according to any of embodiments 7 to 9, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.
11. The use or method according to any of embodiments 7 to 10, wherein the compound of formula (I) is present in an amount in the range of ≥0.01 wt. % to ≤70.0 wt. %, based on the total weight of the composition.
12. A compound of the general formula (I), according to embodiment 2

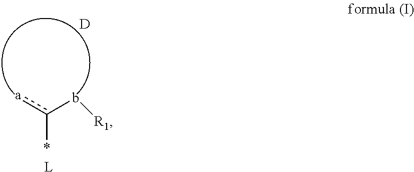

wherein
L is selected from the group consisting of

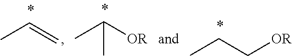

the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula (I) has 1, 2 or 3 —OR groups.
13. The compound according to embodiment 12 wherein R is methyl or ethyl.
14. The compound according to embodiment 12, which is selected from the group consisting of:
1-ethoxymethyl-4-(1-ethoxy-1-methylethyl)cyclohexene;
2-ethoxy-4-(1-ethoxy-1-methylethyl)-1-methylenecyclohexane;
4-isopropenyl-1,2-diethoxy-1-methylcyclohexane;
4-isopropenyl-1,1-diethoxy-2-methylcyclohexane;
6-ethoxy-4-(1-ethoxy-1-methylethyl)-1-methylcyclohexene;
6-ethoxy-4-(2-ethoxy-1-methylethyl)-1-methylcyclohexene;
6-ethoxy-4-(1-ethoxymethylvinyl)-1-methylcyclohexene;
5-ethoxy-1-(2-ethoxy-1-methylethyl)-4-methylcyclohexa-1,3-diene;
1-ethoxymethyl-4-(1-ethoxy-1-methylethyl)benzene;
3-ethoxy-6-(1-ethoxy-1-methylethyl)-3-methylcyclohexene;
3-ethoxy-3-ethoxymethyl-6-(1-ethoxy-1-methylethyl)cyclohexene;
4-(1-ethoxy-1-methylethyl)cyclohex-2-en-1-one;
4-isopropenyl-1,2-dimethoxy-1-methylcyclohexane;
4-isopropenyl-1,1-dimethoxy-2-methylcyclohexane;
6-methoxy-4-(2-methoxy-1-methylethyl)-1-methylcyclohexene;
6-methoxy-4-(1-methoxymethylvinyl)-1-methylcyclohexene;
5-ethoxy-1-(2-methoxy-1-methylethyl)-4-methylcyclohexa-1,3-diene;
3-methoxy-6-(1-methoxy-1-methylethyl)-3-methylcyclohexene;
3-methoxy-3-methoxymethyl-6-(1-ethoxy-1-methylethyl)cyclohexene;
1-ethoxy-3-isopropenyl-6-methyl-cyclohexene;
1-ethoxy-4-isopropenyl-1-methyl-cyclohexane;
3-ethoxy-4-isopropenyl-1-methyl-cyclohexene;
1-(ethoxymethyl)-4-isopropenyl-cyclohexene;
1-(diethoxymethyl)-3-isopropenyl-1-methyl-cyclopentane;
1,2-diethoxy-4-isopropenyl-1-methyl-cyclohexane; and
4-(1-methoxy-1-methylethyl)cyclohex-2-en-1-one.
   15. A composition comprising at least one compound of the formula (I) according to any of embodiments 12 to 14, and
      (i) at least one aroma chemical that is different from the compound of formula (I), or
      (ii) at least one non-aroma chemical carrier, or
      (iii) both of (i) and (ii).
   16. The composition according to embodiment 15, wherein the at least one non-aroma chemical carrier (ii) is selected from the group consisting of surfactants, oil components, antioxidants, deodorant-active agents and solvents.
   17. The composition according to embodiment 15 or 16, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.
   18. A process for preparing a compound of formula (I) according to embodiment 12, comprising subjecting mono-, di- or tri-unsaturated, non-aromatic, monocyclic terpene hydrocarbon to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.
   19. The process according to embodiment 18, wherein the monocyclic terpene is selected from the group consisting of limonene, α-phellandrene, β-phellandrene, α-terpinene, β-terpinene, γ-terpinene, terpinolene.
   20. A compound of the general formula (II), according to embodiment 2

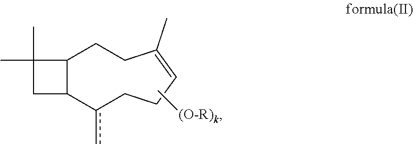

where [symbol] is a C—C single bond or a C═C double bond,
k is 1, 2 or 3;
R is unsubstituted, linear or branched C₁-C₈-alkyl and preferably unsubstituted, linear C₁-C₄-alkyl and the OR groups are bonded to any carbon atoms which are not part of a C═C double bond.
21. The compound according to embodiment 20, wherein R is methyl or ethyl.
22. The compound of the general formula (II) according to embodiment 20, which is selected from the group consisting of:
3-ethoxy-4,11,11-trimethyl-8-methylenebicyclo[7,2,0]undec-4-ene;
6-ethoxy-4,11,11-trimethyl-8-methylenebicyclo[7,2,0]undec-4-ene;
7-ethoxy-4,11,11-trimethyl-8-methylenebicyclo[7,2,0]undec-4-ene;
9-ethoxy-4,11,11-trimethyl-8-methylenebicyclo[7,2,0]undec-4-ene;
4-ethoxymethyl-11,11-dimethyl-8-methylenebicyclo[7,2,0]undec-4-ene;
6,8-diethoxy-4,8,11,11-tetramethylbicyclo[7,2,0]undec-4-ene:
8-(diethoxymethyl)-6-ethoxy-4,11,11-trimethylbicyclo[7,2,0]undec-4-ene;
3-methoxy-4,11,11-trimethyl-8-methylenebicyclo[7,2,0]undec-4-ene;
6-methoxy-4,11,11-trimethyl-8-methylenebicyclo[7,2,0]undec-4-ene;
7-methoxy-4,11,11-trimethyl-8-methylenebicyclo[7,2,0]undec-4-ene;
9-methoxy-4,11,11-trimethyl-8-methylenebicyclo[7,2,0]undec-4-ene;
4-methoxymethyl-11,11-dimethyl-8-methylenebicyclo[7,2,0]undec-4-ene;
6,8-dimethoxy-4,8,11,11-tetramethylbicyclo[7,2,0]undec-4-ene; and
8-(dimethoxymethyl)-6-methoxy-4,11,11-trimethylbicyclo[7,2,0]undec-4-ene.
   23. Use of a compound of the general formula (II), according to embodiment 2

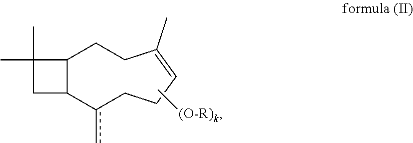

where [symbol] is a C—C single bond or a C═C double bond,
k is 1, 2 or 3;
R is unsubstituted, linear or branched C₁-C₈-alkyl and preferably unsubstituted, linear C₁-C₄-alkyl and the OR groups are bonded to any carbon atoms which are not part of a C═C double bond
as an aroma chemical, preferably as a fragrance.
24. The use according to embodiment 23, wherein R is methyl or ethyl.
25. Method of imparting an aroma impression, preferably a fragrance impression to a composition comprising
   at least the step of adding a compound of formula (II) according to embodiments 20 to 22 to a composition.
26. The use or method according to embodiments 23 to 25, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.
27. The use or method according to embodiments 23 to 26, wherein the compound of formula (II) is present in an amount in the range of ≥0.01 wt. % to ≤70.0 wt. %, based on the total weight of the composition.
28. A composition comprising at least one compound of the formula (II) according to embodiments 20 to 22, and
   (i) at least one aroma chemical that is different from the compound of formula (II), or
   (ii) at least one non-aroma chemical carrier, or
   (iii) both of (i) and (ii).
29. The composition according to embodiment 28, wherein the at least one non-aroma chemical carrier (ii) is selected from the group consisting of surfactants, oil components, anti-oxidants, deodorant-active agents and solvents.
30. The composition according to any of embodiments 28 to 29, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.
31. A process for preparing the compound of formula (II) according to embodiment 20, comprising subjecting a mono-, di- or tri-unsaturated, non-aromatic, bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.
32. The process according to embodiment 31, wherein the bicyclic terpene hydrocarbon is selected from the group consisting of α-pinene, β-pinene, camphene, 3-carene, β-caryophyllene.

### EXAMPLES

The present invention is illustrated in detail by non-restrictive working examples which follow. More particularly, the test methods specified hereinafter are part of the general disclosure of the application and are not restricted to the specific working examples.

### Example A: Electrolysis

### Abbreviations

CI: Chemical ionization

EI: Electron ionization

Area %: Area as %

GC: Gas chromatography

GCMS: Gas chromatography coupled with mass spectrometer

MS: Mass spectrometry

MKUS: Graphite type from SGL

MTBS: Methyl-tri-n-butylammonium methylsulfate

MTES: Methyltriethylammonium methylsulfate

HEMS: Tris(2-hydroxyethyl)methylammonium methylsulfate

BMP-TFSi: N-n-Butyl-N-methylpyrrolidinium bis(trifluoromethylsulfonyl)imide

Et₄NOTs: Tetraethylammonium tosylate

EMIM-EtSO₄: Ethylmethylimidazolium ethylsulfate

EMIM-OTs: Ethylmethylimidazolium tosylate

Na-MeSO₄: Sodium methylsulfate

In the examples below, the following electrodes were used:
Graphite electrode for stacked plate cells: d=140 mm, h=5 mm, type SIGRAFINE® MKUS, manufacturer SGL
Rod-shaped graphite electrode for tubular cell: d=3.5 cm, h=85 cm, properties, type SIGRAFINE® MKUS, manufacturer SGL
Annular stainless steel cathode for tubular cell: size d (internal)=4.5 cm, h=85 cm, V2A steel, type, Feinmechanik BASF
Boron-doped diamond electrode rod for tubular cell: size d=3.5 cm, h=85 cm, properties, type DIACHEM®, 15 ym BDD on silicon (multilayer), manufacturer CONDIAS
Graphite electrode for glass beaker cell: 70×20×5 mm (5 cm immersion depth), properties, type SIGRAFINE® MKUS, manufacturer SGL.

Examples 1 and 2 below were carried out in a stacked plate cell using bipolar-arranged graphite electrodes (MKUS, 10 gaps). The gap width between the plates was about 1 mm. A constant MSP galvanostat from GOSSEN served as current source.

Examples 3, 4, 7 and 12 were carried out in a tubular cell having an internal diameter of 4.5 cm with a central rod-shaped anode (length 85 cm, diameter 3.5 cm) and cylindrically arranged stainless steel cathode (length 85 cm, internal diameter 4.5 cm). The electrode spacing was about 10 mm. A constant SSP galvanostat from GOSSEN served as current source.

Examples 5, 6, 8 to 11 and 13 were in a glass beaker cell having a volume of 100 ml with two electrodes (area 10 cm²) mounted with a 1 cm gap. A constant MSP Galvanostat from GOSSEN served as current source.

The crude output was analyzed by GC/MS or GC/GCMS.

For the gas chromatographic analysis of the electrolysis crude output, the stationary phase used was a DB1 column from Agilent with 60 m length, 0.32 mm diameter and 5 μm layer thickness. This column is heated from 100° C. to 280° C. by means of a temperature program of 6° C./min over 30 min. This temperature is maintained for 20 min. The carrier gas used was helium at a flow rate of 3 mL/min.

Structures were assigned to the GC peaks by means of mass spectrometry (instruments: Gas chromatograph: HP 6890, mass spectrometer: HP 5973, EI 70 eV, C (NH₃)).

### Example 1: Electrolysis of β-pinene

73.3 g (3% by weight) of β-pinene and 115.0 g (5% by weight) of MTBS were dissolved in 2099 g (92% by weight) of ethanol. The solution was electrolyzed in a stacked plate cell with bipolar-arranged graphite electrodes (MKUS, 10 gaps) at 25° C. and standard pressure. In this case, 4 F/mol of pinene were applied at a constant current density of 20 mA/cm², which provided clamping voltages of 34.2 to 51.4 V. 2312.8 g of crude output were obtained at a conversion of pinene of 80%.

According to GC/GCMS, the crude output comprised the following products in fractions of >5%:
2-ethoxy-4-(1-methylvinyl)-1-methylenecyclohexane (13.5 area %);
1-ethoxymethyl-4-(1-methylvinyl)cyclohexene (11.1 area %);
2-ethoxy-4-(1-ethoxy-1-methylethyl)-1-methylenecyclohexane (7.5 area %);
1-ethoxymethyl-4-(1-ethoxy-1-methylethyl)cyclohexene (21.4 area %).

### Example 2: Electrolysis of β-pinene

230 g (9% by weight) of β-pinene and 231.25 g (5% by weight) of tris (2-hydroxyethyl) methylammonium methylsulfate were dissolved in 2099 g (92% by weight) of ethanol. The solution was electrolyzed in a stacked plate cell with bipolar-arranged graphite electrodes (MKUS, 10 gaps) at 25° C. and standard pressure. In this case, 4 F/mol of pinene were applied at a constant current density of 20 mA/cm², which provided clamping voltages of 44.8 to 48.4 V. 2312.8 g of crude output were obtained at a conversion of pinene of 96%.

According to GC/GCMS, the crude output comprised the following products in fractions of >5%:
2-ethoxy-4-(1-methylvinyl)-1-methylenecyclohexane (7.5 area %);
1-ethoxymethyl-4-(1-methylvinyl)cyclohexene (16.7 area %);
2-ethoxy-4-(1-ethoxy-1-methylethyl)-1-methylenecyclohexane (9.4 area %);
1-ethoxymethyl-4-(1-ethoxy-1-methylethyl)benzene (5.9 area %);
1-ethoxymethyl-4-(1-ethoxy-1-methylethyl)cyclohexene (24.6 area %).

### Example 3: Electrolysis of D-(+)-limonene

108.8 g (5% by weight) of D-(+)-limonene and 115.0 g (5% by weight) of MTBS were dissolved in 2099 g (92% by weight) of ethanol. The solution was electrolyzed in a tubular cell with a graphite anode and stainless-steel cathode at 25° C. and standard pressure. In this case, 4 F/mol of D-(+)-limonene were applied at a constant current density of 20 mA/cm², which provided clamping voltages of 8.4 to 8.7 V. 2159 g of crude output were obtained at a conversion of D-(+)-limonene of 97%.

According to GC/GCMS, the crude output comprised the following main products:
5-isopropenyl-2-methylcyclohexanone;
2-ethoxy-4-(1-methylvinyl)-1-methylenecyclohexane.

### Example 4: Electrolysis of D-(+)-limonene

108.8 g (5% by weight) of D-(+)-limonene and 115.0 g (5% by weight) of MTBS were dissolved in 2099 g (92% by weight) of ethanol. The solution was electrolyzed in a tubular cell with a boron-doped diamond anode and stainless-steel cathode at 25° C. and standard pressure. In this case, 4 F/mol of D-(+)-limonene were applied at a constant current density of 20 mA/cm², which provided clamping voltages of 9.5 to 10.8 V. 2227 g of crude output were obtained at a conversion of D-(+)-limonene of 39%.

According to GC/GCMS, the crude output comprised the following main products:
5-isopropenyl-2-methylcyclohexanone;
2-ethoxy-4-(1-methylvinyl)-1-methylenecyclohexane.

### Example 5: Electrolysis of α-terpinene

3.0 g (4% by weight) of α-terpinene and 3.5 g (5% by weight) of MTBS were dissolved in 70 g (91% by weight) of ethanol. The solution was electrolyzed in a glass beaker cell with graphite electrodes at 25° C. and standard pressure. In this case, 4 F/mol of α-terpinene were applied at a constant current density of 20 mA/cm², which provided clamping voltages of 10.3 to 13.1 V. 68.2 g of crude output were obtained at a conversion of terpinene of 93%.

According to GC/GCMS, the crude output comprised the following main products:
5-ethoxy-1-(2-ethoxy-1-methylethyl)-4-methylcyclohexa-1,3-diene;
1-ethoxymethyl-4-(1-methylethyl)benzene.

### Example 6: Electrolysis of Terpinolene

2.9 g (4% by weight) of terpinolene and 3.5 g (5% by weight) of MTBS were dissolved in 70 g (91% by weight) of ethanol. The solution was electrolyzed in a glass beaker cell with graphite electrodes at 25° C. and standard pressure. In this case, 4 F/mol of terpinolene were applied at a constant current density of 20 mA/cm², which provided clamping voltages of 11.7 to 13.4 V. 73.7 g of crude output were obtained at a conversion of terpinolene of 93%.

According to GC/GCMS, the crude output comprised the following products in fractions >5 area %:
1-ethoxymethyl-4-(1-methylethyl)benzene;
5-ethoxy-1-(1-methylethyl)-4-methylcyclohexa-1,3-diene;
1-ethoxymethyl-4-(1-ethoxy-1-methylethyl)cyclohexene;
5-ethoxy-1-(2-ethoxy-1-methylethyl)-4-methylcyclohexa-1,3-diene;
6-ethoxy-4-(1-ethoxymethylvinyl)-1-methylcyclohexene.

### Example 7: Electrolysis of β-carophyllene

108.8 g (5% by weight) of β-carophyllene and 115.0 g (5% by weight) of MTBS were dissolved in 2099 g (92% by weight) of ethanol. The solution was electrolyzed in a tubular cell with a graphite anode and stainless-steel cathode at 25° C. and standard pressure. In this case, 4 F/mol of β-carophyllene were applied at a constant current density of 20 mA/cm², which provided clamping voltages of 8.0 to 8.7 V. 2221 g of crude output were obtained at a conversion of β-carophyllene of 83%.

According to GC/GCMS, the crude output comprised the following main products:
6-ethoxy-4,11,11-trimethyl-8-methylenebicyclo[7,2,0]undec-4-ene;
6,8-diethoxy-4,8,11,11-tetramethylbicyclo[7,2,0]undec-4-ene; and
8-(diethoxymethyl)-6-ethoxy-4,11,11-trimethylbicyclo[7,2,0]undec-4-ene.

### Example 8: Electrolysis of α-pinene

108.8 g (5% by weight) of α-pinene and 115.0 g (5% by weight) of MTBS were dissolved in 2099 g (92% by weight) of ethanol. The solution was electrolyzed in a tubular cell with a graphite anode and stainless-steel cathode at 25° C. and standard pressure. In this case, 4 F/mol of pinene were applied at a constant current density of 20 mA/cm², which provided clamping voltages of 8.0 to 8.6 V. 2176 g of crude output were obtained at a conversion of α-pinene of 97%.

According to GC/GCMS, the crude output comprised the following main products:
6-ethoxy-4-(1-methylvinyl)-1-methylcyclohexene;
6-ethoxy-4-(1-ethoxy-1-methylethyl)-1-methylcyclohexene;
6-ethoxy-4-(2-ethoxy-1-methylethyl)-1-methylcyclohexene;
1-ethoxy-6,6-bis(ethoxymethyl)-2-methylbicyclo[3.1.1]cycloheptene.

### Example 9: Electrolysis of 3-carene

3.5 g (5% by weight) of 3-carene and 3.5 g (5% by weight) of MTBS were dissolved in 70 g (90% by weight) of ethanol. The solution was electrolyzed in a glass beaker cell with graphite electrodes at 25° C. and standard pressure. In this case, 4 F/mol of 3-carene were applied at a constant current density of 20 mA/cm², which provided clamping voltages of 12.7 to 14.9 V. 67.2 g of crude output were obtained at a conversion of 3-carene of 98%.

According to GC/GCMS, the crude output comprised in fractions >3 area %:
3 monounsaturated carene monoethyl ether isomers (m/z 180);
2 monounsaturated carene diethyl ether isomers (m/z 226);
2 monounsaturated carene triethyl ether isomers (m/z 270).

### Example 10: Electrolysis of Camphene

2.9 g (5% by weight) of camphene and 2.5 g (4% by weight) of tris (2-hydroxyethyl) methylammonium methylsulfate were dissolved in 55.2 g (91% by weight) of ethanol. The solution was electrolyzed in a glass beaker cell with graphite electrodes at 25° C. and standard pressure. In this case, 4 F/mol of camphene were applied at a constant current density of 20 mA/cm², which provided clamping voltages of 14.5 to 17.0 V. 64.0 g of crude output was obtained at a conversion of camphene of 92%.

According to GC/GCMS, the crude output comprised in fractions >5 area %:
4 monounsaturated camphene diethyl ether isomers (m/z 226);
1 monounsaturated camphene triethyl ether isomer (m/z 270).

### Example 11: Electrolysis of α-phellandrene

3.0 g (4% by weight) of α-phellandrene and 3.5 g (5% by weight) of MTBS were dissolved in 70 g (91% by weight) of ethanol. The solution was electrolyzed in a glass beaker cell with graphite electrodes at 25° C. and standard pressure. In this case, 4 F/mol of phellandrene were applied at a constant current density of 20 mA/cm², which provided clamping voltages of 8.7 to 13.9 V. 70.4 g of crude output were obtained at a conversion of α-phellandrene of 95%.

According to GC/GCMS, the crude output comprised the following main products in fractions >5 area %:
1 monounsaturated carene monoethyl ether isomer (m/z 180);
3 monounsaturated carene diethyl ether isomers (m/z 226).

### Example 12: Electrolysis of S-(−)-limonene

108.8 g (5% by weight) of S-(−)-limonene and 191.7 g (8% by weight) of MTBS were dissolved in 2099 g (87% by weight) of methanol. The solution was electrolyzed in a tubular cell with a graphite anode and stainless-steel cathode at 25° C. and standard pressure. In this case, 2 F/mol of S-(−)-limonene were applied at a constant current density of 20 mA/cm², which provided clamping voltages of 8.0 to 8.6 V. 2328.3 g of crude output were obtained at a conversion of S-(−)-limonene of 84%.

According to GC/GCMS, the crude output comprised in fractions >5 area %:
2-methoxy-4-(1-methylvinyl)-1-methylenecyclohexane;
1-methoxymethyl-4-(1-methylvinyl)cyclohexene;
3-methoxy-3-methyl-6-(1-methylvinyl)cyclohexene;
6-methoxy-4-(1-methylvinyl)-1-methylcyclohexene;
1-methoxy-1-methyl-4-(1-methylvinyl)cyclohexane;
1,1-dimethoxy-2-methyl-4-(1-methylvinyl)cyclohexane.

### Examples 13a-13h: Electrolysis of D-(+)-limonene

2.8 g (5% by weight) of D-(+)-limonene and 2.5 g (4% by weight) of the respective conductive salt were dissolved in 55.2 g (91% by weight) of ethanol. The solution was electrolyzed in a glass beaker cell with graphite electrodes at 25° C. and standard pressure. In this case, 4 F/mol of phellandrene were applied at a constant current density of 20 mA/cm². The conductive salt used, and the conversion are stated in the Table.

According to GC/GCMS, the crude output comprised the product distribution specified in the Table I below.

| | | A | B | C | D | E | F | G | H | I | J | K | L |
|---|---|---|---|---|---|---|---|---|---|---|---|---|---|
| | RT [min] | 27.82 | 28.10 | 28.65 | 28.79 | 29.10 | 29.21 | 29.43 | 29.74 | 30.26 | 32.06 | 33.18 | 33.61 |
| | Conduc- | | | | | | | | | | | | |
| | tive salt | | | | | | | | | | | | |
| 13a | MTBS | 21.39 | 22.45 | 0.00 | 8.03 | 2.46 | 0.00 | 5.24 | 2.81 | 0.30 | 0.00 | 9.37 | 7.25 |
| 13b | MTES | 0.00 | 27.06 | 9.40 | 0.00 | 0.00 | 3.35 | 9.93 | 4.21 | 0.81 | 0.00 | 1.76 | 11.63 |
| 13c | Et₄NOTs | 0.00 | 21.12 | 2.60 | 4.68 | 1.01 | 2.40 | 20.06 | 3.61 | 3.80 | 4.74 | 6.25 | 4.67 |
| 13d | NaMeSO₄ | 0.00 | 20.29 | 10.75 | 3.84 | 3.95 | 9.41 | 14.90 | 4.86 | 2.71 | 2.77 | 8.68 | 5.94 |
| 13e | EMIM | 0.00 | 27.80 | 4.83 | 7.18 | 0.00 | 4.85 | 14.81 | 4.77 | 2.12 | 2.92 | 9.54 | 6.88 |
| | EtSO₄ | | | | | | | | | | | | |
| 13f | HEMS | 0.00 | 30.87 | 6.89 | 9.56 | 0.00 | 0.27 | 5.03 | 4.07 | 0.32 | 0.00 | 10.20 | 9.95 |
| 13g | EMIM- | 0.00 | 19.72 | 2.50 | 3.01 | 3.04 | 8.72 | 21.07 | 3.90 | 4.49 | 4.77 | 5.98 | 4.20 |
| | OTs | | | | | | | | | | | | |
| 13h | BMP-TFSi | 0.00 | 11.41 | 5.58 | 0.00 | 0.00 | 5.70 | 14.67 | 2.04 | 0.62 | 0.00 | 26.37 | 9.22 |

A: methyl(1-methylvinyl)cyclohexanone (isomer of C)
B: 2-ethoxy-4-(1-methylvinyl)-1-methylenecyclohexane
C: 2-methyl-5-(1-methylvinyl)cyclohexanone
D: 2-ethoxy-4-(1-methylvinyl)-1-methylcyclohexene
E: limonene ethyl ether isomer of unknown structure
F: limonene ethyl ether isomer of unknown structure
G: ethoxy-(1-methylvinyl)-1-methylcyclohexene (isomer of D)
H: limonene ethyl ether isomer of unknown structure
I: ethoxy-(1-methylvinyl)-1-methylcyclohexene (isomer of B or D)
J: ethoxy-(1-methylvinyl)-1-methylcyclohexene (isomer of B or D)
K: ethoxy-(1-methylvinyl)-1-methylcyclohexene (isomer of B or D)
L: limonene ethyl ether isomer of unknown structure

### Example 14: Electrolysis of D-(+)-limonene

122.5 g D-(+)-limonene (purity 94%; 5 weight %) and 294.0 g MTBS (12 weight %) were dissolved in 2035.5 g ethanol (83 weight %). The solution was electrolyzed at 25° C. in a capillary gap cell with a stack of bipolar electrodes. Graphite (MKUS) electrodes as anodes covered with steel foil (0,025 mm) as cathodes were employed resulting in 9 gaps. 3F were applied at a constant current density of 17 mA/cm². 2414 g of crude output were obtained.

### Example 15: Electrolysis of S-(−)-limonene

122.5 g S-(−)-limonene (purity 95%; 5 weight %) and 294.0 g MTBS (12 weight %) were dissolved in 2035.5 g ethanol (83 weight %). The solution was electrolyzed at 25° C. in a capillary gap cell with a stack of bipolar electrodes. Graphite (MKUS) electrodes as anodes covered with a steel cathode (2 mm) were employed resulting in 7 gaps. 3F were applied at a constant current density of 20 mA/cm². 2393.6 g of crude output were obtained.

Isolation and characterization of compound of example 14.

Pre-Separation of the Sample

An aliquot of the sample was pre-separated by means of column chromatography. Silica gel 60 was used as stationary phase. The mobile phase consists out of n-hexane (A) and methylene chloride (B) in different mixing ratios. Six fractions were collected, consisting out of approximately 100 mL, each, resp. 200 mL for the last fraction (100% A; 80% A+20% B; 60% A+40% B; 40% A+60% B; 20% A+80% B; 100% B). The solvent of each fraction was removed under a nitrogen stream.

Preparative High-Performance Liquid Chromatography (HPLC)

The pre-separated fractions were further analyzed by means of preparative HPLC. The HPLC system used was a Young Lin Instrument (Anyang-si, South Korea) YL9110S Quaternary Pump (flow: 15 mL min⁻¹) with different combinations of eluents (Table A). The HPLC was equipped with a polar column (guard column: Macherey-Nagel (Dren, Germany), Nucleodur 100-5, 10 mm×16 mm, preparative column: Macherey-Nagel, Nucleodur 100-5, 250 mm×21 mm) coupled with a YL9120S UVNis Detector (wavelengths: 210 and 235 nm), and an Advantec (Dublin, Calif.) CHF 112 SC fraction collector (Refer table A for solvent system used and the isolates collected). Every fraction resulting from preparative HPLC was analyzed by means of gas chromatography coupled with a flame ionization detector (G-FID) (Table B,).

Pure fractions were combined, the solvent was removed, the residue was dissolved in CDCl₃, and the samples were analyzed by nuclear magnetic resonance (NMR) spectroscopy. The NMR-experiments (¹H, ¹³C, DEPT135, COSY, HSQC, HMBC) were performed on Bruker (Rheinstetten, Germany) Avance II 400 MHz, Bruker Avance III HD 400 MHz and Bruker Avance III 600 MHz spectrometers (Table C).

Olfactory Impression

The olfactory impression (quality and intensity) of the compounds prepared were tested using scent strip tests. For this purpose, strips of absorbent paper were dipped into a solution containing 1 to 10 wt % of the compound to be tested in ethanol. After evaporation of the solvent (about 30 s) the olfactory impression was evaluated by a panel of five trained perfumers. Refer table C for the odor impression.

TABLE A
Solvents used for the isolation of the
compounds by means of preparative HPLC

| | | eluents used for prep. HPLC (n-hexane = A, |
|---|---|---|
| isolate | pre-separated | methylene chloride = B, tert-butyl methyl |
| no. | fraction | ether = C) |
| 1a | 3 | solvents: A + B |
| | | 0 min 100% A, 15 min 97% A, 30 min 90% A, |
| | | 50 min 70% A, 60 min 50% A |
| 1b | 3 | solvents: A + B |
| | | 0 min 95% A, 20 min 95% A |
| 2a | 4 | solvents: A + C |
| | | 0 min 100% A, 30 min 98.75% A, 40 min 87.5% |
| | | A, 50 min 75% A, 60 min 75% A |
| 2b | 4 | solvents: A + B |
| | | 0 min 100% A, 45 min 65% A, 55 min 40% A, |
| | | 60 min 0% A |
| 3a | 6 | see 2b |
| 3b | 6 | solvents: A + B |
| | | 0 min 100% A, 20 min 97% A, 30 min 94% A, |
| | | 40 min 90% A, 50 min 75% A, 55 min 50% A, |
| | | 60 min 25% A |
| 4a | 6 | solvents: A + B |
| | | 0 min 100% A, 20 min 96% A, 30 min 92% A, |
| | | 50 min 75% A, 60 min 60% A, 65 min 45% A, |
| | | 70 min 25% A, 75 min 0% A |
| 4b | 5/6 | solvents: A + B |
| | | 0 min 100% A, 20 min 95% A, 30 min 90% A, |
| | | 45 min 75% A, 55 min 50% A, 60 min 25% A |
| 5 | 5 | solvents: A + B |
| | | 0 min 100% A, 48 min 0% A, 52 min 0% A |
| 6 | 5 | see 2b |
| 7 | 6 | see 4a |
| 8a | 6 | see 2a |
| 8b | 6 | solvents: A + B + C |
| | | 0 min 99% A 0% B, 60 min 50% A 50% B |
| 9a | 5 | solvents: A + B + C |
| | | 0 min 100% A, 60 min 2% A 96% B |
| 9b | 6 | solvents: A + B |
| | | 0 min 90% A, 20 min 80% A, 40 min 55% A, 55 |
| | | min 20% A, 60 min 0% A, 75 min 0% A |
| 10a | 6 | see 9a |
| 10b | 6 | see 9a |

TABLE B
GC parameters used for analysis of preparative HPLC fractions

| | |
|---|---|
| gas chromatograph | Agilent 7890A |
| column | Agilent HP-InnoWAX |
| | 30 m × 320 μm ID, 0.25 μm film thickness |
| carrier gas | hydrogen (5.0) |
| gas flow | 2.0 mL · min⁻¹ (constant) |
| autosampler | Agilent autosampler G2614A |
| inlet | split/splitless, 250° C. |
| injection volume | 1 μL |
| split ratio | 1:20 |
| temperature program | 40° C. (3 min), 20° C. · min⁻¹ to 240° C. |
| | (7 min), |
| detector | FID |
| detector temperature | 250° C. |
| detector gases | hydrogen, 40 mL · min⁻¹ |
| | air, 400 mL · min⁻¹ |
| | nitrogen, 25 mL · min⁻¹ |

TABLE C
Compound identification by GC-MS and NMR spectroscopy and their Odor
impression

| Compound | | | Odor |
|---|---|---|---|
| no. | NMR/GC | Structure | impression |
| 1a | ¹H NMR (CDCl₃, 400 MHz): δ 4.75 (1 H, m), 4.73 (1 H, m), 4.44 (1 H, d, J = 3 Hz), 3.69 (2 H, m), 2.82 (1 H, m), 2.24 (1 H, m), 1.86 (1 H, m), 1.76 (1 H, m), 1.72 (3 H, br. s), 1.36 (1 H, m), 1.28 (3 H, t, J = 7 Hz), 1.26 (1 H, m), 1.07 (3 H, d, J = 7 Hz) ¹³C NMR (CDCl₃, 100 MHz): δ 158.7, 150.2, 109.8, 97.0, 61.8, 42.6, 32.4, 30.2, 26.3, 20.7, 18.8, 14.7 GC-MS (El, 70 eV): 180 (100) [M^{•+}], 165 (99), 43 (68), 137 (63), 109 (61), | 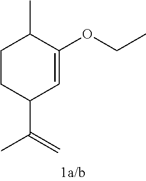 | herbal, earthy, fresh carrot like |
| | 95 (60), 81 (56), 123 (50), 41 (38), 67 | | |
| | (37) | | |
| 1b | ¹H NMR (CDCl₃, 400 MHz): δ 4.76 (1 | | herbal, |
| | H, m), 4.74 (1 H, m), 4.42 (1 H, d, J = | | earthy, |
| | 3 Hz), 3.70 (2 H, m), 2.83 (1 H, m), | | fresh |
| | 2.21 (1 H, sext, J = 6 Hz), 1.73 (1 H, | | carrot like |
| | m), 1.72 (3 H, br. s), 1.61 (1 H, m), | | |
| | 1.49 (1 H, m), 1.47 (1 H, m), 1.28 (3 | | |
| | H, t, J = 7 Hz), 1.09 (3 H, d, J = 7 Hz) | | |
| | ¹³C NMR (CDCl₃, 100 MHz): δ 159.1, | | |
| | 150.3, 109.8, 96.9, 61.7, 42.9, 31.8, | | |
| | 29.2, 24.6, 20.4, 19.1, 14.7 | | |
| | GC-MS (El, 70 eV): 180 (100) [M^{•+}], | | |
| | 165 (99), 43 (68), 109 (64), 137 (63), | | |
| | 95 (62), 81 (57), 123 (51), 41 (38) 67 | | |
| | (38) | | |
| 2a | ¹H NMR (CDCl₃, 600 MHz): δ 4.86 (1 H, m), 4.81 (1 H, m), 4.70 (2 H, m), 3.87 (1 H, t, J = 3 Hz), 3.43 (1 H, dq, J = 7/10 Hz), 3.29 (1 H, dq, J = 7/10 Hz), 2.53 (1 H, tt, J = 3/12 Hz), 2.33 (1 H, tdt, J = 2/5/13 Hz), 2.18 (1 H, m), 2.06 (1 H, m), 1.87 (1 H, m), 1.73 (3 H, br. s), 1.48 (1 H, ddd, J = 3/11/13 Hz), 1.27 (1 H, m), 1.20 (3 H, t, J = 7 Hz) ¹³C NMR (CDCl₃, 150 MHz): δ 149.8, | 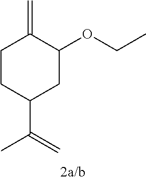 | herbal, fresh, green, dill like |
| | 148.0, 110.6, 108.6, 79.0, 62.6, 38.6, | | |
| | 38.2, 32.9, 30.4, 21.0, 15.4 | | |
| | GC-MS (El, 70 eV): 134 (100), 119 | | |
| | (68), 91 (59), 93 (44), 137 (37), 41 | | |
| | (32), 83 (32), 79 (30), 67 (27), 92 | | |
| | (26), 180 (1) [M^{•+}] | | |
| 2b | ¹H NMR (CDCl₃, 400 MHz): δ 4.95 (1 | | herbal, |
| | H, q, J = 2 Hz), 4.76 (1 H, q, J = 2 | | spicy, |
| | Hz), 4.70 (2 H, m), 3.68 (1 H, m), 3.60 | | earthy, |
| | (2 H, m), 2.42 (1 H, ddd, J = 3/4/13 | | juniper |
| | Hz), 2.17 (1 H, m), 2.15 (1 H, m), 2.02 | | like |
| | (1 H, m), 1.79 (1 H, m), 1.71 (3 H, br. | | |
| | s), 1.27 (1 H, m), 1.24 (1 H, m), 1.23 | | |
| | (3 H, t, J = 7 Hz) | | |
| | ¹³C NMR (CDCl₃, 100 MHz): δ 148.9, | | |
| | 148.8, 109.1, 104.5, 79.6, 65.0, 44.4, | | |
| | 39.7, 34.1, 33.1, 20.6, 15.6 | | |
| | GC-MS (El, 70 eV): 137 (100), 93 | | |
| | (98), 91 (91), 119 (90), 83 (76), 79 | | |
| | (75), 134 (73), 41 (71), 67 (67), 55 | | |
| | (65), 180 (11) [M^{•+}] | | |
| 3a | ¹H NMR (CDCl₃, 400 MHz): δ 5.66 (1 H, m), 5.66 (1 H, m), 4.76 (1 H, m), 4.70 (1 H, m), 3.43 (2 H, qq, J = 7/9 Hz), 2.75 (1 H, m), 1.90 (1 H, m), 1.86 (1 H, m), 1.72 (3 H, br. s), 1.62 (1 H, m), 1.51 (1 H, m), 1.26 (3 H, s), 1.17 (3 H, t, J = 7 Hz) ¹³C NMR (CDCl₃, 100 MHz): δ 148.1, 133.4, 132.0, 110.5, 73.4, 57.2, 43.1, 32.2, 26.6, 25.9, 20.9, 16.4 GC-MS (El, 70 eV): 165 (100), 137 (60), 134 (60), 93 (52), 43 (51), 91 (47), 109 (47), 107 (46), 79 (38), 77 | 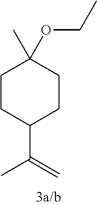 | sweetish, fruity, anise like, licorice like |
| | (33), 180 (1) [M^{•+}] | | |
| 3b | ¹H NMR (CDCl₃, 400 MHz): δ 5.69 (1 | | spicy, |
| | H, m), 5.69 (1 H, m), 4.79 (1 H, m), | | anise like, |
| | 4.75 (1 H, m), 3.43 (2 H, qd, J = 2/7 | | cinnamon |
| | Hz), 2.65 (1 H, m), 1.94 (1 H, m), 1.74 | | like, clove |
| | (3 H, m), 1.71 (2 H, m), 1.40 (1 H, m), | | like |
| | 1.24 (3 H, s), 1.15 (3 H, t, J = 7 Hz) | | |
| | ¹³C NMR (CDCl₃, 100 MHz): δ 148.1, | | |
| | 133.0, 132.6, 110.8, 71.9, 57.4, 43.1, | | |
| | 32.6, 26.6, 24.6, 21.0, 16.4 | | |
| | GC-MS (El, 70 eV): 165 (100), 93 | | |
| | (93), 137 (92), 107 (84), 91 (73), 94 | | |
| | (72), 43 (72), 109 (67), 79 (62), 77 | | |
| | (49), 180 (1) [M^{•+}] | | |
| 4a | ¹H NMR (CDCl₃, 400 MHz): δ 5.48 (1 H, m), 4.80 (1 H, m), 4.76 (1 H, m), 3.84 (1 H, m), 3.58 (1 H, dq, J = 7/9 Hz), 3.47 (1 H, dq, J = 7/9 Hz), 2.22 (1 H, ddd, J = 3/8/11 Hz), 2.01 (1 H, m), 1.88 (1 H, m), 1.77 (3 H, br. s), 1.72 (1 H, m), 1.68 (3 H, br. s), 1.61 (1 H, dddd, J = 5/10/11/13 Hz), 1.17 (3 H, t, J = 7 Hz) ¹³C NMR (CDCl₃, 100 MHz): δ 147.5, 137.4, 122.6, 110.6, 77.1, 63.7, 46.8, | 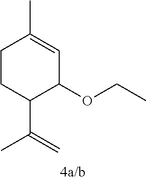 | herbal, green, floral, fresh carrot like |
| | 29.8, 26.6, 23.3, 21.0, 15.7 | | |
| | GC-MS (El, 70 eV): 112 (100), 97 | | |
| | (89), 83 (80), 84 (42), 108 (35), 91 | | |
| | (19), 41 (18), 77 (14), 79 (12), 55 | | |
| | (11), 180 (1) [M^{•+}] | | |
| 4b | ¹H NMR (CDCl₃, 600 MHz): δ 5.51 (1 | | herbal, |
| | H, m), 4.72 (2 H, m), 3.91 (1 H, m), | | earthy, |
| | 3.64 (1 H, dq, J = 7/9 Hz), 3.46 (1 H, | | fresh |
| | dq, J = 7/9 Hz), 2.23 (1 H, m), 2.19 (1 | | carrot like |
| | H, m), 2.03 (1 H, m), 1.93 (1 H, m), | | |
| | 1.73 (3 H, br. s), 1.72 (3 H, m), 1.46 | | |
| | (1 H, ddd, J = 10/12/13 Hz), 1.21 (3 | | |
| | H, t, J = 7 Hz) | | |
| | ¹³C NMR (CDCl₃, 150 MHz): δ 149.3, | | |
| | 135.5, 124.4, 108.9, 78.0, 63.8, 40.8, | | |
| | 34.4, 31.0, 20.4, 19.2, 15.7 | | |
| | GC-MS (El, 70 eV): 84 (100), 112 | | |
| | (72), 134 (70), 119 (53), 55 (52), 79 | | |
| | (42), 41 (39), 95 (38), 77 (38), 83 | | |
| | (38), 180 (13) [M^{•+}] | | |
| 5 | ¹H NMR (CDCl₃, 600 MHz): δ 5.70 (1 H, m), 4.71 (2 H, m), 3.84 (2 H, s), 3.44 (2 H, q, J = 7 Hz), 2.16 (1 H, m), 2.15 (1 H, m), 2.10 (2 H, m), 1.97 (1 H, m), 1.84 (1 H, m), 1.74 (3 H, br. s), 1.48 (1 H, m), 1.21 (3 H, t, J = 7 Hz) ¹³C NMR (CDCl₃, 150 MHz): δ 150.0, 134.9, 124.0, 108.6, 75.0, 65.2, 41.2, 30.5, 27.5, 26.5, 20.8, 15.2 GC-MS (El, 70 eV): 93 (100), 91 (99), 119 (76), 79 (70), 137 (64), 67 (61), | 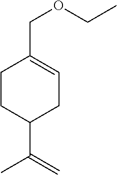 | fruity, sweetish, green, coriander like |
| | 83 (53), 92 (50), 68 (49), 134 (47), | | |
| | 180 (16) [M^{•+}] | | |
| 6 | ¹H NMR (CDCl₃, 600 MHz): δ 5.60 (1 H, m), 4.73 (2 H, m), 3.68 (1 H, dq, J = 7/9 Hz), 3.60 (1 H, m), 3.43 (1 H, dq, J = 7/9 Hz), 2.36 (1 H, m), 2.15 (1 H, m), 2.05 (1 H, m), 1.81 (1 H, m), 1.77 (1 H, m), 1.74 (3 H, br. s), 1.40 (1 H, ddd, J = 4/13/14 Hz), 1.23 (3 H, t, J = 7 Hz) ¹³C NMR (CDCl₃, 150 MHz): δ 149.9, 133.2, 125.5, 108.6, 76.0, 64.8, 35.5, | 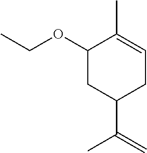 | herbal, earthy, fresh carrot like, parsley like |
| | 32.1, 31.1, 21.0, 21.0, 15.8 | | |
| | GC-MS (El, 70 eV): 137 (100), 84 | | |
| | (94), 91 (76), 119 (66), 109 (63), 93 | | |
| | (61), 55 (47), 77 (46), 112 (45), 83 | | |
| | (42), 180 (18) [M^{•+}] | | |
| 7 | ¹H NMR (CDCl₃, 600 MHz): δ 4.69 (1 H, m), 4.65 (1 H, m), 4.11 (1 H, s), 3.81 (1 H, m), 3.81 (1 H, m), 3.54 (1 H, m), 3.54 (1 H, m), 2.52 (1 H, m), 1.96 (1 H, ddd, J = 1/8/13 Hz), 1.75 (1 H, m), 1.75 (1 H, m), 1.72 (3 H, br. s), 1.50 (1 H, m), 1.37 (1 H, m), 1.22 (3 H, t, J = 7 Hz), 1.22 (3 H, t, J = 7 Hz), 1.11 (1 H, dd, J = 11/13 Hz), 1.03 (3 H, s) | 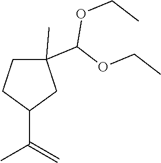 [SMILES: C=C(C)C1CCC(C)(C(OCC)OCC)C1] | tart, woody, earthy, spicy |
| | ¹³C NMR (CDCl₃, 150 MHz): δ 149.0, | | |
| | 110.72, 107.8, 65.9, 65.7, 47.4, 47.2, | | |
| | 41.0, 35.4, 30.7, 24.6, 21.3, 15.6, | | |
| | 15.5 | | |
| | GC-MS (El, 70 eV): 103 (100), 75 | | |
| | (40), 47 (33), 99 (13), 107 (9), 93 (8), | | |
| | 43 (8), 71 (8), 41 (7), 55 (7), 226 (1) | | |
| | [M^{•+}] | | |
| 8a | ¹H NMR (CDCl₃, 400 MHz): δ 4.76 (1 H, m), 4.73 (1 H, m), 2.45 (1 H, dt, J = 2/11 Hz), 2.38 (1 H, m), 2.36 (1 H, m), 2.28 (1 H, m), 2.13 (1 H, m), 1.94 (1 H, m), 1.74 (3 H, br. s), 1.65 (1 H, m), 1.38 (1 H, qd, J = 4/13 Hz), 1.04 (3 H, d, J = 7 Hz) ¹³C NMR (CDCl₃, 100 MHz): δ 212.7, 147.7, 109.6, 47.0, 46.9, 44.8, 34.9, 30.8, 20.5, 14.4 GC-MS (El, 70 eV): 67 (100), 95 (79), 68 (52), 81 (48), 82 (46), 109 (42), 41 | 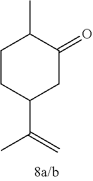 | fresh, minty, herbal, tart, caraway like |
| | (40), 69 (36), 55 (28), 39 (27), 152 | | |
| | (18) [M^{•+}] | | |
| 8b | ¹H NMR (CDCl₃, 400 MHz): δ 4.83 (1 | | fresh, |
| | H, m), 4.69 (1 H, m), 2.60 (1 H, m), | | minty, |
| | 2.55 (1 H, m), 2.42 (1 H, m), 2.40 (1 | | herbal, |
| | H, m), 1.85 (1 H, m), 1.85 (2 H, m), | | tart |
| | 1.73 (3 H, br. s), 1.60 (1 H, m), 1.09 | | |
| | (3 H, d, J = 7 Hz) | | |
| | ¹³C NMR (CDCl₃, 100 MHz): δ 214.0, | | |
| | 146.9, 111.5, 44.6, 44.1, 44.0, 30.7, | | |
| | 26.4, 26.4, 21.5, 15.6 | | |
| | GC-MS (El, 70 eV): 67 (100), 95 (92), | | |
| | 68 (52), 82 (45), 41 (41), 69 (37), 81 | | |
| | (36), 152 (31) [M^{•+}], 55 (29), 39 (27) | | |
| 9a | ¹H NMR (CDCl₃, 400 MHz): δ 4.70 (1 H, m), 4.68 (1 H, m), 3.61 (1 H, dq, J = 7/9 Hz), 3.37 (1 H, m), 3.37 (2 H, m), 3.23 (1 H, br. s), 2.18 (1 H, tt, J = 2/12 Hz), 1.73 (1 H, m), 1.72 (3 H, br. s), 1.67 (1 H, m), 1.53 (1 H, m), 1.44 (1 H, m), 1.37 (1 H, m), 1.26 (1 H, m), 1.17 (3 H, t, J = 7 Hz), 1.16 (3 H, t, J = 7 Hz), 1.16 (3 H, s) ¹³C NMR (CDCl₃, 100 MHz): δ 150.8, 108.1, 79.8, 74.8, 64.7, 55.5, 37.7, 30.2, 29.6, 26.3, 21.5, 20.9, 16.1, 15.7 | 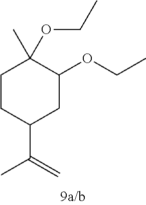 [SMILES: C=C(C)C1CCC(C)(OCC)C(OCC)C1] | herbal, dill like, earthy |
| | GC-MS (El, 70 eV): 99 (100), 71 (36), | | |
| | 43 (19), 58 (11), 140 (8), 108 (7), 100 | | |
| | (7), 86 (6), 41 (6), 93 (6), 226 (1) [M^{•+}] | | |
| 9b | ¹H NMR (CDCl₃, 400 MHz): δ 4.73 (1 | | fresh, |
| | H, m), 4.69 (1 H, m), 3.69 (1 H, dq, J = | | menthol |
| | 7/9 Hz), 3.50 (1 H, dq, J = 7/9 Hz), | | like, |
| | 3.40 (1 H, m), 3.40 (1 H, m), 2.98 (1 | | floral, |
| | H, m), 1.97 (1 H, m), 1.96 (1 H, m), | | citrus like |
| | 1.76 (2 H, m), 1.73 (3 H, br. s), 1.42 | | |
| | (2 H, m), 1.23 (3 H, s), 1.18 (3 H, t, J = | | |
| | 7 Hz), 1.17 (3 H, t, J = 7 Hz), 1.12 | | |
| | (1 H, m) | | |
| | ¹³C NMR (CDCl₃, 100 MHz): δ 149.8, | | |
| | 108.6, 85.0, 74.6, 65.4, 56.6, 44.6, | | |
| | 34.6, 31.2, 26.2, 21.5, 20.6, 16.2, | | |
| | 15.6 | | |
| | GC-MS (El, 70 eV): 99 (100), 71 (38), | | |
| | 43 (19), 58 (10), 100 (7), 140 (7), 108 | | |
| | (6), 86 (6), 41 (6), 93 (6), 226 (1) [M^{•+}] | | |
| 10a | ¹H NMR (CDCl₃, 400 MHz): δ 4.73 (1 H, m), 4.70 (1 H, m), 2.95 (1 H, m), 2.49 (1 H, m), 2.17 (3 H, s), 2.04 (1 H, m), 1.93 (1 H, m), 1.84 (1 H, m), 1.84 (1 H, m), 1.73 (3 H, br. s), 1.61 (1 H, ddd, J = 10/11/12 Hz), 1.49 (1 H, m) ¹³C NMR (CDCl₃, 100 MHz): δ 210.6, 147.4, 109.0, 51.9, 47.7, 31.2, 30.5, 28.8, 27.3, 21.0 GC-MS (El, 70 eV): 43 (100), 109 (99), 67 (88), 137 (49), 71 (39), 41 (28), 79 (26), 93 (26), 55 (26), 82 | 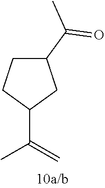 | minty, tart, floral, fruity, fresh, citrus like |
| | (25), 152 (24) [M^{•+}] | | |
| 10b | ¹H NMR (CDCl₃, 400 MHz): δ 4.70 (2 | | minty, |
| | H, m), 3.00 (1 H, dtd, J = 5/8/10 Hz), | | tart, |
| | 2.47 (1 H, m), 2.16 (3 H, s), 2.06 (1 H, | | resinous |
| | dddd, J = 1/5/8/13 Hz), 1.98 (1 H, m), | | like, |
| | 1.88 (1 H, m), 1.78 (1 H, m), 1.73 (3 H, | | green, |
| | br. s), 1.65 (1 H, dt, J = 10/13 Hz), 1.48 | | spicy |
| | (1 H, dtd, J = 8/10/12 Hz) | | |
| | ¹³C NMR (CDCl₃, 100 MHz): δ 210.9, | | |
| | 148.0, 108.7, 51.3, 46.4, 32.9, 31.7, | | |
| | 28.9, 28.5, 21.5 | | |
| | GC-MS (El, 70 eV): 71 (100), 109 (99), | | |
| | 43 (78), 67 (67), 137 (65), 152 (44) | | |
| | [M^{•+}], 41 (26), 39 (23), 79 (22), 82 (21) | | |

### ADVANTAGEOUS COMPOSITIONS

The compounds formed in example 1 to 15 were formulated in the compositions according to tables 2 and 3 and were labelled as “compound A”.

TABLE 2
Compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl)oxybutan-2-ol) | 4 | 4 |
| Ethyl-2-methyl butyrate | 4 | 4 |
| Geranyl acetate | 5 | 5 |
| Helional (3-(1,3-benzodioxol-5-yl)-2-methylpropanal) | 10 | 10 |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b-tetrahydroindeno[1,2- | 15 | 15 |
| d][1,3]dioxine) | | |
| Verdox (2-tert-butylcyclohexyl) acetate) | 25 | 25 |
| Bergamot oil bergaptene free | 25 | 25 |
| Linalol | 30 | 30 |
| Dipropylene glycol | 45 | 45 |
| Iso E Super (Tetramethyl acetyloctahydronaphthalenes) | 110 | 110 |
| Pyranol (4-methyl-2-(2-methylpropyl)oxan-4-ol) | 170 | 170 |
| Hedione (methyl 3-oxo-2-pentylcyclopentaneacetate) | 200 | 200 |
| Galaxolide 50% IPM (1,3,4,6,7,8-hexahydro-4,6,6,7,8,8- | 300 | 300 |
| hexamethylcyclopenta(g)-2-benzopyran 50% in isopropyl | | |
| myristate) | | |
| Compound A | 20 | 50 |
| | 1020 | 1050 |

TABLE 3
Compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% 2-methyl-3-(p- | 10 | 10 |
| isopropylphenyl)-propionaldehyde; secondary component: | | |
| 5% 3-(p-cumenyl)-2-methylpropionic acid) | | |
| Bicyclononalactone (3,4,4a,5,6,7,8,8a-octahydrochromen- | 10 | 10 |
| 2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept-2- | 30 | 30 |
| yl]cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane-5,17-dione) | 170 | 170 |
| Galaxolide 50% IPM (1,3,4,6,7,8-hexahydro-4,6,6,7,8,8- | 200 | 200 |
| hexamethylcyclopenta(g)-2-benzopyran 50% in isopropyl | | |
| myristate) | | |
| Compound A | 20 | 50 |
| | 1020 | 1050 |

Composition according to table 2 and table 3 namely 1A, 1B, 2A,2B could be included in various compositions selected from the group consisting of Deo pump spray, Clean hair-conditioner, Face wash gel, Foam bath concentrate, Hair gel, Self-foaming bodywash, Sprayable sun care emulsion, Sprayable sun protection emulsion, Emollient facial gel, 2-phases oil foam bath, Shampoos, Shower bath, Hydro-alcoholic AP/Deo pump spray, Aerosol, Aqueous/alcoholic AP/Deo roll-on, Styling Gel Type “Out of Bed”, Shaving Foam, Sensitive skin Baby shampoo, Body wash for Sensitive Skin, Gloss Enhancing Shampoo for Sensitive Scalp, Deo Stick, Baby Wipe, After shave balm, Face Gel, Face Day Care Cream, Face Cleanser, Body lotion, Sun Care SPF50+, Sprayable Lotion, Hand dish cleaner—regular, Hand dish cleaner—concentrate, Sanitary cleaner—concentrate, All-purpose cleaner, Anti-bacterial fabric softener, Detergent composition, Powder detergent composition and Liquid detergent composition.

A person skilled in art may be well versed with the various general formulations for the above-mentioned products.

Compositions 1A, 1B, 2A and 2B can for example be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A or 2B.

1.-32. (canceled)

33. A method for preparing C₁-C₈-alkyl ethers of mono- and bicyclic terpenes, comprising subjecting a mono-, di- or tri-unsaturated, non-aromatic, mono- or bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

34. The method according to claim 33, wherein the C₁-C₈-alkyl ethers of mono- and bicyclic terpenes is selected from at least one compound of the 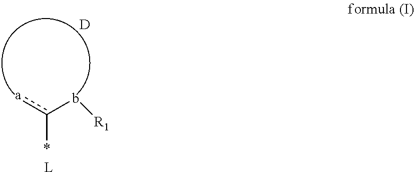
wherein
L is selected from the group consisting of 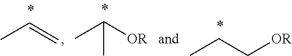
and the point of attachment of L is indicated by *,
D is selected from the group consisting of
—CH₂—C(CH₃)OR—CH(OR)—; —CH₂—C(CH₃)OR—CH═; —CH₂—C(CH₃)═CH—;
—CH₂—CH(CH₃)—C(OR)═; —CH₂—C(CH₂—O—R)═CH—; —CH₂—C(OR)₂—CH(CH₃)—;
—CH═C(CH₃)—CH(OR)—; —CH═C(CH₃)—CH(OR)—; ═CH—C(CH₃)(OR)—CH₂—;
═CH—C(OR)(CH₂OR)—CH₂—; ═CH—C(═O)—CH₂—; —C(CH₃)(CH(OR)₂)—CH₂—;
—CH2-C(CH3)(OCH3)-CH(OCH3)-; —C═C(CH3)-CH(OCH3)-; —C═C(—CH2OCH3)-CH═; —CH2-C(O)—CH(OCH3)-; —CH2-C(CH2-O—CH3)=CH—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
or 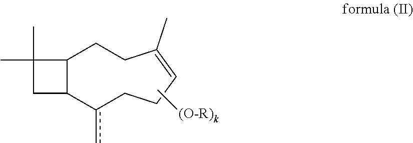
where [symbol] is a C—C single bond or a C═C double bond,
k is 1, 2 or 3;
R is unsubstituted, linear or branched C₁-C₈-alkyl.

35. The method according to claim 33, wherein the terpene hydrocarbon has a menthene or menthadiene skeleton and is selected from limonene, α-phellandrene, β-phellandrene, α-terpinene, β-terpinene, γ-terpinene, terpinolene and mixtures thereof.

36. The method according to claim 33, wherein the terpene hydrocarbon is a bicyclic terpene hydrocarbon which is selected from α-pinene, β-pinene, camphene, 3-carene, β-caryophyllene and mixtures thereof.

37. The method according to claim 33, wherein the C₁-C₈-alkanol is selected from the group consisting of linear C₁-C₄-alkanols.

38. The method according to claim 33 having at least one of the features a) to l):
a) the electrolyte comprises less than 30% by weight of water, based on the total mass of the electrolyte;
b) the concentration of the alkanol in the electrolyte is in the range of 60 to 98% by weight, based on the total mass of the electrolyte;
c) the concentration of the terpene in the electrolyte is in the range of 1 to 25% by weight, based on the total mass of the electrolyte;
d) the electrolyte comprises at least one conductive salt at a concentration in the range of 1 to 20% by weight, based on the total mass of the electrolyte;
e) the conductive salt is selected from salts of which the anions are selected from C₁-C₈-alkyl sulfates, C1-C8-alkyl sulfonates, aryl sulfonates and bis(fluoro-C₁-C₄-alkylsulfonyl)imides;
f) the anode material is a carbon material;
g) the electrolysis is carried out in an undivided electrolysis cell;
h) the electrolysis is carried out galvanostatically;
i) the electrolysis is carried out in an electrolysis cell with a bipolar electrode arrangement;
j) the electrolysis is carried out in a bipolar stacked plate cell;
k) the electrolysis is carried out with a quantity of electricity of 0.3 to 10 F per mol of terpene;
l) the electrolysis is carried out with a current density in the range of 5 to 80 mA/m2.

39. The use of a compound of the general formula (I), according to claim 34 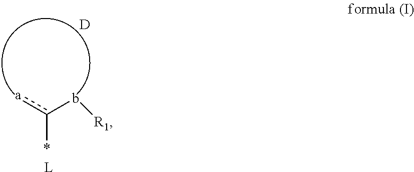
wherein
L is selected from the group consisting of 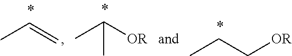
and the point of attachment of L is indicated by *,
D is selected from the group consisting of
—CH₂—C(CH₃)OR—CH(OR)—;
—CH₂—C(CH₃)OR—CH═;
—CH₂—C(CH₃)═CH—;
—CH₂—CH(CH₃)—C(OR)═;
—CH₂—C(CH₂—O—R)═CH—;
—CH₂—C(OR)₂—CH(CH₃)—;
—CH═C(CH₃)—CH(OR)—;
—CH═C(CH₃)—CH(OR)—;
═CH—C(CH₃)(OR)—CH₂—;
═CH—C(OR)(CH₂OR)—CH₂—;
═CH—C(═O)—CH₂—;
—C(CH₃)(CH(OR)₂)—CH₂—;
—CH₂—C(CH₃)(OCH₃)—CH(OCH₃)—;
C═C(CH₃)—CH(OCH₃)—;
C═C(—CH₂OCH₃)—CH═;
—CH₂—C(O)—CH(OCH₃)—;
—CH₂—C(CH₂—O—CH₃)═CH—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups,
as an aroma chemical.

40. The use according to claim 39, wherein R is methyl or ethyl.

41. A method of imparting an aroma impression to a composition comprising at least the step of adding a compound as claimed in claim 39 to a composition.

42. The use or method according to claim 39, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.

43. The use or method according to claim 39, wherein the compound of formula (I) is present in an amount in the range of ≥0.01 wt. % to ≤70.0 wt. %, based on the total weight of the composition.

44. A compound of the general formula (I), according to claim 34 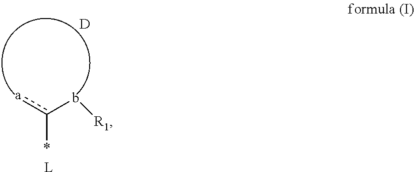
wherein
L is selected from the group consisting of 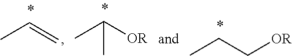
and i the point of attachment of L is indicated by *,
D is selected from the group consisting of
—CH₂—C(CH₃) OR—CH(OR)—;
—CH₂—C(CH₃) OR—CH═;
—CH₂—C(CH₃)═CH—;
—CH₂—CH(CH₃)—C(OR)═,
—CH₂—C(CH₂—O—R)═CH—,
—CH₂—C(OR)₂—CH(CH₃)—,
—CH═C(CH₃)—CH(OR)—,
—CH═C(CH₃)—CH(OR)—,
═CH—C(CH₃)(OR)—CH₂—,
═CH—C(OR)(CH₂OR)—CH₂—,
═CH—C(═O)—CH₂—,
—C(CH₃)(CH(OR)₂)—CH₂—,
wherein “a” and “b” denote carbon atoms and are linked via the above carbon chain D to form a 5- or 6-membered ring,
R is unsubstituted, linear or branched C₁-C₈-alkyl, and
R₁ is H or —OR,
wherein the compound of formula I has 1, 2 or 3 —OR groups.

45. The compound according to claim 44 wherein R is methyl or ethyl.

46. The compound according to claim 44, which is selected from the group consisting of:
1-ethoxymethyl-4-(1-ethoxy-1-methylethyl)cyclohexene;
2-ethoxy-4-(1-ethoxy-1-methylethyl)-1-methylenecyclohexane;
4-isopropenyl-1,2-diethoxy-1-methylcyclohexane;
4-isopropenyl-1,1-diethoxy-2-methylcyclohexane;
6-ethoxy-4-(1-ethoxy-1-methylethyl)-1-methylcyclohexene;
6-ethoxy-4-(2-ethoxy-1-methylethyl)-1-methylcyclohexene;
6-ethoxy-4-(1-ethoxymethylvinyl)-1-methylcyclohexene;
5-ethoxy-1-(2-ethoxy-1-methylethyl)-4-methylcyclohexa-1,3-diene;
1-ethoxymethyl-4-(1-ethoxy-1-methylethyl)benzene;
3-ethoxy-6-(1-ethoxy-1-methylethyl)-3-methylcyclohexene;
3-ethoxy-3-ethoxymethyl-6-(1-ethoxy-1-methylethyl)cyclohexene;
4-(1-ethoxy-1-methylethyl)cyclohex-2-en-1-one;
4-isopropenyl-1,2-dimethoxy-1-methylcyclohexane;
4-isopropenyl-1,1-dimethoxy-2-methylcyclohexane;
6-methoxy-4-(2-methoxy-1-methylethyl)-1-methylcyclohexene;
6-methoxy-4-(1-methoxymethylvinyl)-1-methylcyclohexene;
5-ethoxy-1-(2-methoxy-1-methylethyl)-4-methylcyclohexa-1,3-diene;
3-methoxy-6-(1-methoxy-1-methylethyl)-3-methylcyclohexene;
3-methoxy-3-methoxymethyl-6-(1-ethoxy-1-methylethyl)cyclohexene;
1-ethoxy-3-isopropenyl-6-methyl-cyclohexene;
1-ethoxy-4-isopropenyl-1-methyl-cyclohexane;
3-ethoxy-4-isopropenyl-1-methyl-cyclohexene;
1-(ethoxymethyl)-4-isopropenyl-cyclohexene;
1-(diethoxymethyl)-3-isopropenyl-1-methyl-cyclopentane;
1,2-diethoxy-4-isopropenyl-1-methyl-cyclohexane; and
4-(1-methoxy-1-methylethyl)cyclohex-2-en-1-one.

47. A composition comprising at least one compound of the formula (I) according to claim 44, and
(i) at least one aroma chemical that is different from the compound of formula (I), or
(ii) at least one non-aroma chemical carrier, or
(iii) both of (i) and (ii).

48. The composition according to claim 47, wherein the at least one non-aroma chemical carrier (ii) is selected from the group consisting of surfactants, oil components, antioxidants, deodorant-active agents and solvents.

49. The composition according to claim 47, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.

50. A process for preparing a compound of formula (I) according to claim 44, comprising subjecting mono-, di- or tri-unsaturated, non-aromatic, monocyclic terpene hydrocarbon to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

51. The process according to claim 50, wherein the monocyclic terpene is selected from the group consisting of limonene, α-phellandrene, β-phellandrene, α-terpinene, β-terpinene, γ-terpinene, terpinolene.

52. A compound of the general formula (II), according to claim 34 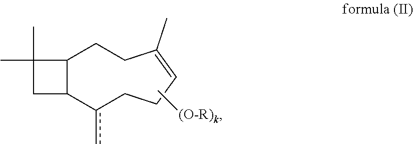
where [symbol] is a C—C single bond or a C═C double bond,
k is 1, 2 or 3;
R is unsubstituted, linear or branched C₁-C₈-alkyl.

53. The compound according to claim 52, wherein R is methyl or ethyl.

54. The compound of the general formula (II) according to claim 52, which is selected from the group consisting of:
3-ethoxy-4,11,11-trimethyl-8-methylenebicyclo[7,2,0]undec-4-ene;
6-ethoxy-4,11,11-trimethyl-8-methylenebicyclo[7,2,0]undec-4-ene;
7-ethoxy-4,11,11-trimethyl-8-methylenebicyclo[7,2,0]undec-4-ene;
9-ethoxy-4,11,11-trimethyl-8-methylenebicyclo[7,2,0]undec-4-ene;
4-ethoxymethyl-11,11-dimethyl-8-methylenebicyclo[7,2,0]undec-4-ene;
6,8-diethoxy-4,8,11,11-tetramethylbicyclo[7,2,0]undec-4-ene;
8-(diethoxymethyl)-6-ethoxy-4,11,11-trimethylbicyclo[7,2,0]undec-4-ene;
3-methoxy-4,11,11-trimethyl-8-methylenebicyclo[7,2,0]undec-4-ene;
6-methoxy-4,11,11-trimethyl-8-methylenebicyclo[7,2,0]undec-4-ene;
7-methoxy-4,11,11-trimethyl-8-methylenebicyclo[7,2,0]undec-4-ene;
9-methoxy-4,11,11-trimethyl-8-methylenebicyclo[7,2,0]undec-4-ene;
4-methoxymethyl-11,11-dimethyl-8-methylenebicyclo[7,2,0]undec-4-ene;
6,8-dimethoxy-4,8,11,11-tetramethylbicyclo[7,2,0]undec-4-ene; and
8-(dimethoxymethyl)-6-methoxy-4,11,11-trimethylbicyclo[7,2,0]undec-4-ene.

55. The use of a compound of the general formula (II), according to claim 34 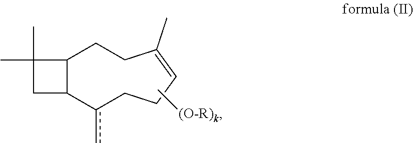
where [symbol] is a C—C single bond or a C═C double bond,
k is 1, 2 or 3;
R is unsubstituted, linear or branched C₁-C₈-alkyl as an aroma chemical.

56. The use according to claim 55, wherein R is methyl or ethyl.

57. A method of imparting an aroma impression to a composition comprising at least the step of adding a compound of formula (II) according to claim 52 to a composition.

58. The use or method according to claim 55, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.

59. The use or method according to claim 55, wherein the compound of formula (II) is present in an amount in the range of ≥0.01 wt. % to ≤70.0 wt. %, based on the total weight of the composition.

60. A composition comprising at least one compound of the formula (II) according to claim 52, and
(i) at least one aroma chemical that is different from the compound of formula (II), or
(ii) at least one non-aroma chemical carrier, or
(iii) both of (i) and (ii).

61. The composition according to claim 60, wherein the at least one non-aroma chemical carrier (ii) is selected from the group consisting of surfactants, oil components, anti-oxidants, deodorant-active agents and solvents.

62. The composition according to claim 60, wherein the composition is selected from the group consisting of perfume compositions, body care compositions, hygiene articles, cleaning compositions, textile detergent compositions, compositions for scent dispensers, foods, food supplements, pharmaceutical compositions and crop protection compositions.

63. A process for preparing the compound of formula (II) according to claim 52, comprising subjecting a mono-, di- or tri-unsaturated, non-aromatic, bicyclic terpene hydrocarbon having 10 to 15 carbon atoms to electrolysis in an electrolyte consisting of up to at least 50% of at least one C1-C8-alkanol and comprising at least one conductive salt, wherein the electrolyte comprises less than 1000 ppm of halide ions and wherein the conductive salt is selected from alkali metal salts and quaternary ammonium salts, of which the anions are selected from the group consisting of organosulfates, organosulfonates, organophosphates, fluoroalkyl carboxylates and disulfonylimides.

64. The process according to claim 63, wherein the bicyclic terpene hydrocarbon is selected from the group consisting of α-pinene, β-pinene, camphene, 3-carene, β-caryophyllene.
